(19) United States
(12) Patent Application Publication
(10) Pub. No.: US 2019/0362816 A1
(43) Pub. Date: Nov. 28, 2019

(54) Systems and Methods for Developing Covalent Inhibitor Libraries for Screening Using Virtual Docking and Experimental Approaches

(71) Applicant: Northwestern University, Evanston, IL (US)

(72) Inventor: Alexander V. Statsyuk, Evanston, IL (US)

(73) Assignee: Northwestern University, Evanston, IL (US)

(21) Appl. No.: 16/470,021

(22) PCT Filed: Dec. 15, 2017
(86) PCT No.: PCT/US2017/066616

§ 371 Date: Jun. 14, 2019

Related U.S. Application Data
(60) Provisional application No. 62/435,347, filed on Dec. 16, 2016.

(51) Int. Cl. G16C 20/64; G16B 15/30; G16C 20/62; A61K 31/195; C40B 40/14; G16C 60/00

(52) U.S. Cl. CPC G16C 20/64; G16B 15/30; G16C 60/00; A61K 31/195; C40B 40/14; G16C 20/62

(57) ABSTRACT

Disclosed are methods and systems for screening covalent ligand libraries to identify potential covalent inhibitors. The methods and systems may also be used to generate a covalent inhibitor library from natural ligands. These covalent inhibitors bind to the receptor irreversibly after initial reversible binding. The covalent inhibitor identified or designed using the present methods may specifically bind to and covalently modify a receptor.

### CROSS-REFERENCE TO RELATED APPLICATIONS

This patent document claims priority under 35 U.S.C. § 119(e) to the U.S. Provisional Patent Application No. 62/435,347, filed Dec. 16, 2016. This Provisional U.S. Application is incorporated herein by reference in its entirety.

### STATEMENT REGARDING FEDERALLY-SPONSORED RESEARCH

The present application was made with government support under 5R01GM15632-02, awarded by the National Institutes of Health. Thus, the government has certain rights in the present application.

### FIELD OF THE INVENTION

The present disclosure generally relates to methods and systems for identifying and ranking highly active covalent inhibitors for a receptor. The disclosed methods and systems may automatically estimate the covalent binding ability of the covalent inhibitors. In other aspects, inhibitory activities of the covalent inhibitors may be further experimentally qualified and/or quantified to refine the hits generated from the methods and systems. Finally, a method to create a library of covalent inhibitors for covalent docking is disclosed.

### BACKGROUND OF THE INVENTION

Generally speaking, there are three types of inhibitors. The first type is a classic inhibitor, where the inhibitor molecule binds to a receptor and prevents other molecules from binding to the receptor (1). More specifically, classic inhibitors physically block a binding site on the receptor or otherwise cause the receptor to change its shape, which prevents other molecules from binding to the receptor. Classic inhibitors may not permanently bind to the receptor, i.e., they associate to the receptor and dissociate from the receptor reversibly. Classic inhibitors typically rely on one or more of hydrogen bonding or Van der Waals interactions to perform or enable the attachment and/or detachment. The second type of inhibitor is a known as a “covalent inhibitor.”(2) Covalent inhibitors bind to a receptor in the same way as a classic inhibitor, but instead of disassociating, covalent inhibitors form a covalent, permanent, chemical bond to the receptor. Examples of covalent inhibitors may include EGFR kinase inhibitors Afatinib used to treat lung cancer, and Bruton Tyrosine kinase inhibitor Ibrutinib used to treat B-cell malignancies. Furthermore, in the field of oncology covalent inhibitors are effective against drug-resistant tumors, and in general display more potency at inhibiting tumor growth. The third type of inhibotrs are covalent reversible inhibitors. Such inhibitors can form a covalent bond with the receptor but this covalent bond is reversible (3).

The kinetics of the three types of inhibitors—the classic inhibitor and the covalent inhibitors—are illustrated below. Specifically, the receptor (R) and the inhibitor (I) combine to form the complex R·I, with a rate of k₁, typically known. Such a process is typically known as “docking.” More specifically, the R·I complex may disassociate with a rate constant of k₋₁ to reform R and I (an example of a classic inhibitor). Alternatively, in the case of a covalent inhibitor, the R·I complex forms a covalent bond between R and I to form a new complex R−I, with a rate constant of k₂. Stated differently, once the inhibitor covalently binds to the receptor, it is irreversibly bound to the receptor and cannot be disassociated. There are also cases in which covalent inhibitors may form covalent bond with the receptor but such bond is reversible (i.e. k₋₂). The kinetics of the covalent irreversible inhibitors can be described using the following parameters: Kᵢ=k₁/k₋₁, and k_{obs}=k₂/(1+Kᵢ/[I]).

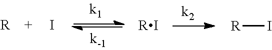

An issue with covalent inhibitors is that they may be too reactive, and in some instances, non-specifically bind to any proteins or receptors the covalent inhibitors encounter (4). In the interaction scheme illustrated above, k₂ is a rate constant for the formation of the covalent receptor-inhibitor complex. Thus, the reactivity of the covalent inhibitor may be influenced by the k₂ rate constant. If k₂ is too large, the covalent inhibitor is considered hyperreactive and will cause non-specific covalent labeling of the receptor, irrespective of reversible binding in k₁ and k₋₁ steps. In this case, if such molecule is converted into a drug, upon administration it will covalently react with many proteins non-specifically causing various side effects. On the other hand, if k₂ is too small then covalent labeling may not occur, and the inhibitor may behave as a conventional reversible inhibitor.

Recently, covalent inhibitors have attracted the attention of major pharmaceutical companies because the use of covalent inhibitors offers an increased potency and extended duration of action when compared to classic reversible inhibitors. Prolonged duration of action translates into lower dosage frequency, i.e., patients have to take fewer pills and do it less frequently. To discover covalent drug leads, one has to screen a library of covalent drugs against the protein receptor and identify those molecules that specifically bind to the receptor and cause its irreversible covalent inhibition. However, screening covalent compound libraries using virtual docking methods, biochemical assays, and phenotypic screens may present challenge because of the danger of non-specific covalent labeling of proteins with hyperreactive compounds, which is caused either by the poor choice of electrophiles which are inherently hyperreactive or the varying structures of drug-like molecules which may cause up to a two-thousandfold increase in the reactivity of otherwise unreactive electrophiles. For example, Pfizer screened its collection of reactive covalent compounds against the HIF-1α receptor and identified eight compounds that covalently labeled the cysteine residue in HIF-1α, as illustrated below (5). Further evaluation of the data showed seven out of eight compounds were false positive hits (88% false positive hit rate). The seven compounds were indiscriminately reacting with random cysteine, lysine, and histidine residues in the HIF-1α receptor, rather than forming covalent bonds with the specific cysteine residue near the drug binding site.

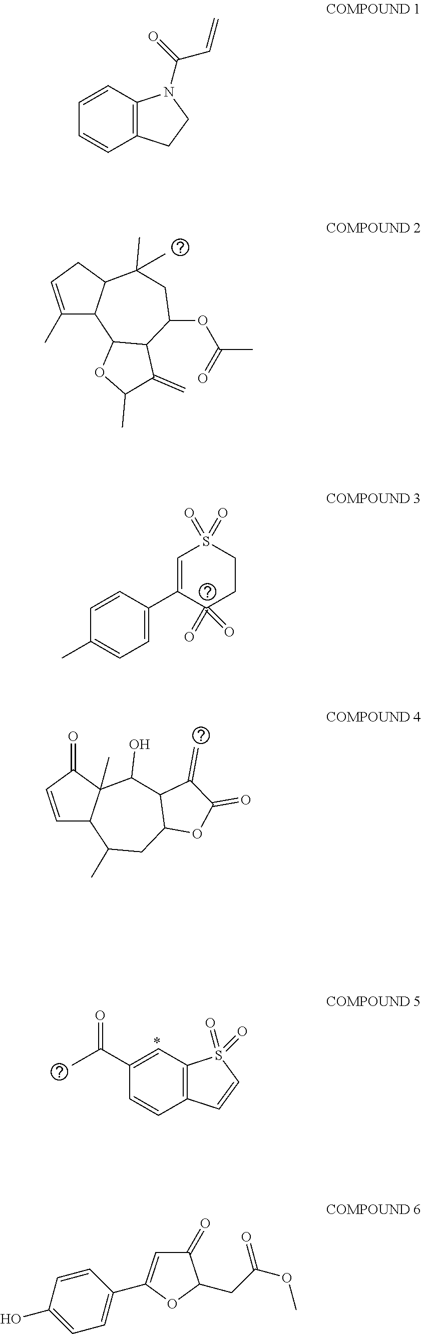

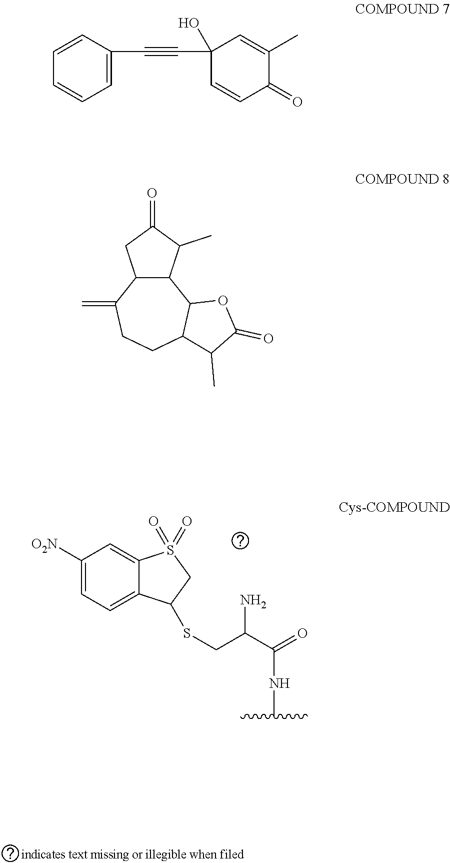 [SMILES: CC1=CC(O)(C#Cc2ccccc2)C=CC1=O ; C=C1CCC2C(C)C(=O)OC2C2C(C)C(=O)CC12 ; *NC(=O)C(N)CSC1CS(=O)(=O)c2cc([N+](=O)[O-])ccc21]

A study by Mann, et al. demonstrates how moderately reactive electrophiles can become hyper-reactive when attached to the drug-like fragment (6). The study reported the screen of reactive cysteine compounds against thymidylate synthase based on acrylamide electrophiles. Amongst the nine compounds used in the screen, one compound, acrylamide, was identified as being hyperreactive and causing non-specific covalent modification of thymidylate synthase, as shown below. The other two acrylamide containing compounds, one of which is illustrated below, however, were not hyperreactive and were identified as a specific covalent modifier and inhibitor of thymidylate synthase. These two examples show that because design rules are lacking, it is challenging to design libraries of covalent inhibitors for screening and to predict their reactivity. One out of three hits is a hyperreactive compound; this translates into 30% false hit rate. The above results demonstrate the ability to identify promising covalent inhibitors is still in its infancy.

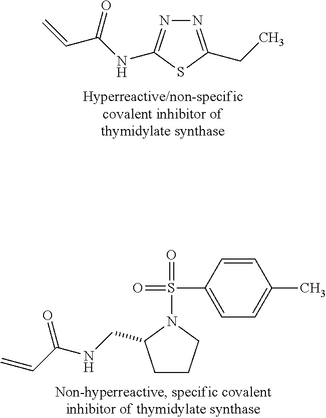

Another difficulty with potential covalent compounds relates to the synthesis of the covalent compounds. Generally, the syntheses of potential covalent compounds involve multiple synthetic and purification steps. When using such syntheses and purification steps, generating libraries of covalent inhibitors for screening purposes or developing a structure-activity relationship (SAR) may be difficult.

There remains a need for methods and systems that automatically generate covalent chemical libraries, either by using methods of organic synthesis or by using novel computer algorithms to generate/synthesize a library of covalent compounds for virtual docking methods. The resulting covalent compounds should not or only minimally cause non-specific covalent labeling of receptors. To achieve these requirements, special design rules need to be implemented to ensure that all covalent compounds in the library do not cause non-specific covalent labeling of the receptor. Subsequently, if desired, the compounds may be synthesized using standard chemical techniques and tested in an assay the results of which may be used to further refine the compounds generated using the algorithm and the SAR.

### SUMMARY OF THE INVENTION

Disclosed herein are methods and systems for identifying potential covalent inhibitors, ranking the identified potential covalent inhibitors, preparing the identified potential covalent inhibitors, and determining the effectiveness and potency of the potential covalent inhibitors.

In one aspect, the methods for identifying potential covalent inhibitors may include providing a potential receptor containing at least one nucleophilic group and a potential covalent inhibitor containing at least one electrophilic group capable of forming a covalent bond with the receptor. The method may also include computer mediated docking the potential covalent inhibitor into the receptor and ranking the docked potential covalent inhibitor based on one or more screening criteria and their combinations thereof (7). The screening criteria may include Van der Waals radius, angle of attack, solvation effects, cone angle, bond length, bond strength, electronic character, and their combinations thereof. The method may continue with selecting the highly ranked potential covalent inhibitors.

The method may include providing three-dimensional structure information of the potential receptor. The method may further include docking the potential covalent inhibitor into a ligand binding region or a ligand binding pocket of the receptor. The ligand binding region or ligand binding pocket of the receptor may be known. The ligand binding region or ligand binding pocket of the receptor may be identified base on the three-dimensional structure information. The method may include docking the potential covalent inhibitor into the receptor is performed using a docking algorithm. The docking algorithm may include GLIDE and CovDock (7).

The method may further include experimentally testing the selected covalent inhibitors. Experimentally testing of the selected covalent inhibitors may include measuring binding kinetics of the potential covalent inhibitor, specified by a two-step mechanism. The potential covalent inhibitor initially binds reversibly to the receptor, then an electrophilic center of the potential covalent inhibitor binds irreversibly to a nucleophile center of an amino acid within the receptor (8,9).

The potential covalent inhibitor may be a carboxylic acid (R₁—COOH) coupled to an electrophilic fragment terminated with an aminomethyl group, or an aminomethyl group (R₁—CH₂—NH₂) coupled to an electrophilic fragment terminated with a carboxylic acid. R₁ may be a drug-like fragment, or a fragment comprising ten to seventeen non-hydrogen atoms.

In another aspect, methods of generating a covalent inhibitor library may include identifying a natural ligand capable of covalently binding to a receptor, wherein the natural ligand comprising a directing group and a reactive group containing an electrophilic fragment. The method may also include modifying the reactive group by replacing the directing group with a carboxylic acid or an amine. The method may continue with coupling the carboxylic acid-modified reactive group to a second directing group comprising an aminomethyl group (R₁—CH₂—NH₂) or coupling the amine-modified reactive group to a third directing group comprising a carboxylic acid.

The electrophilic fragment may be a Michael acceptor or an alkylating agent. The electrophilic fragment may be an sp² or sp Michael acceptor. The electrophilic fragment further comprises an electron withdrawing group, which may be one of nitro, amides, esters, acid, nitriles, ketones, sulfones, sulfoxides, sulfonamides, nitriles, halides, lactams, lactones, oxygen heterocycles, nitrogen heterocycles, substituted or unsubstituted aromatic fragments, and epoxides.

An electrophile center of the potential covalent inhibitor reacts with a nucleophile center of an amino acid in the receptor. The amino acid may be cysteine, methionine, proline, tryptophan, asparagine, glutamine, tryptophan, or histidine The nucleophile center of the amino acid in the receptor may be sulfur or nitrogen.

### BRIEF DESCRIPTION OF DRAWINGS

To facilitate further description of the invention, the following drawings are provided in which:

FIG. 1 is block diagram illustrating a computing environment for automatically provisioning computing resources, according to aspects of the present disclosure;

FIG. 2A illustrates a workflow for coupling fragments containing carboxylic acids with electrophiles; FIG. 2B illustrates a workflow for coupling fragments containing amines with electrophiles;

FIG. 3 is a diagram of a computing system, according to aspects of the present disclosure; and

FIG. 4A-4B illustrate examples of docking poses of identified covalent inhibitors of cysteine protease Cathepsin L.

### DETAILED DESCRIPTION OF THE INVENTION

Aspects of the present disclosure involve methods and systems for automatically identifying potential covalent inhibitors for a receptor. The methods or systems include identifying a potential receptor containing nucleophilic groups and identifying covalent inhibitors for the receptor. The reactive groups of these covalent inhibitors are derived from the natural product, known enzyme inhibitors, or new electrophiles, which are analogs of those present in natural products and known enzyme inhibitors. The system automatically identifies reactive and/or electrophilic groups within the known inhibitor. This is achieved by using common chemistry knowledge and translating that into standard computer language as needed. The system docks, ranks, and selects the identified reactive or electrophilic groups to prepare highly active potential covalent inhibitors for the receptor. These highly ranked inhibitors comprise electrophilic groups that are not hyperreactive and undergo specific covalent reaction with the receptor.

In other aspects, the system may store the identified covalent inhibitors in a library or series of libraries that only contain and/or otherwise identify covalent inhibitors and/or covalent drug-like compounds that do not cause non-specific labeling of proteins. The generated libraries may be screened using virtual docking methods, biomedical assays, and phenotypic screens, and thereby ensure that none of the compounds used in the screening can cause non-specific covalent labeling of proteins with hyperreactive compounds.

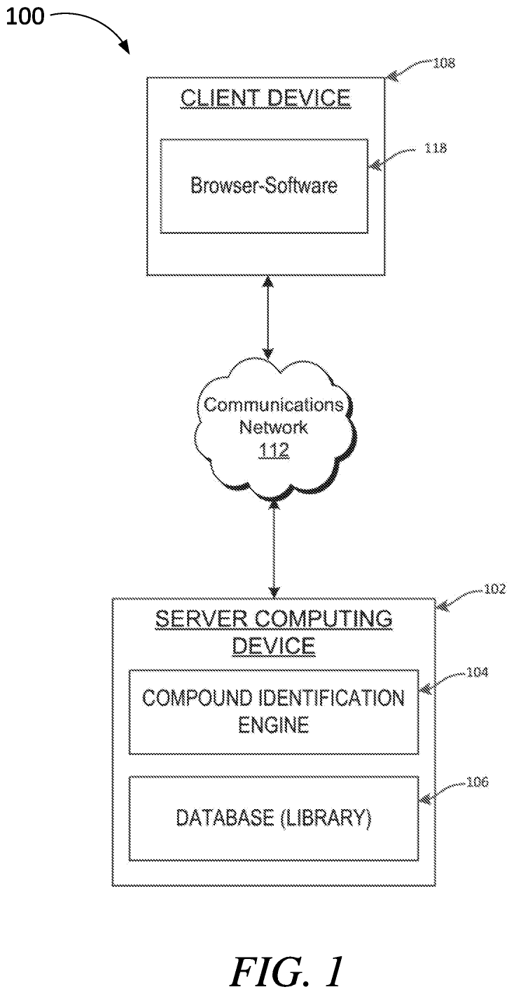

FIG. 1 illustrates one example of a computing architecture 100 that may be used to automatically identify potential covalent inhibitors for a receptor for inclusion into a library for screening using virtual docking methods, biomedical assays, and phenotypic screens, and/or the like. In the illustrated embodiment, the computing architecture 100 includes a server device 102 includes a compound identification engine 104 configured to execute one or more methods, algorithms, and/or computing processes (as described below) to automatically identify covalent inhibitors and/or generate libraries of covalent inhibitors, electrophilic compounds, covalent drugs, and/or the like, for subsequent use in screening.

The server device 102 further includes a database and/or data store 106 (or some other database architecture including those embodied in a single database or multiple databases of the same or differing platforms) that is used to store, among other information, data relating to covalent inhibitors for a receptor, covalent drugs, covalent natural products, electrophilic compounds, and/or the like. In one particular embodiment, the data store 106 may include or otherwise be a library of electrophilic compounds for use in screening to discover covalent drugs. The compound library may be considered dynamic in that the covalent compounds included in the library may change over time, expand and/or contract, and/or the like. All or portions of the library may be screened and/or analyzed using virtual docking methods, biomedical assays, and phenotypic screens, and/or the like.

A client device 108 allows for online communication with the server device 102 through communications network 112, which may be the Internet, an intranet, an Ethernet network, a wired network, a wireless network, and/or another communication network. Additionally, the client network 108 may include network-enabled devices, such as web-browser software 118 for communication over the communications network 112 (e.g., browsing the internet). In one specific embodiment, the client device may be a personal computer, workstation, mobile device, mobile phone, tablet device, processor, and/or other processing device capable of implementing and/or executing processes, software, applications, etc. Additionally, the one or more client device(s) 106 may include one or more processors that process software or other machine-readable instructions and may include a memory to store the software or other machine-readable instructions and data. The computing architecture 100 includes various computing devices, processors, and/or the like that may be used to implement the various methods, algorithms, and/or processes described below.

In one aspect, the method, process and/or algorithm for identifying and selecting highly ranked potential covalent inhibitors comprises (a) identifying a potential receptor containing at least one nucleophilic group; (b) identifying known inhibitors for this receptor; (c) using a computer algorithm to dock potential covalent inhibitors or covalent inhibitor fragments into the potential receptor; (d) analyzing the nucleophilic character of receptor and the electrophilic groups of the potential covalent inhibitor or the covalent inhibitor fragment; (e) identifying the electrophilic portion of the potential covalent inhibitors and/or covalent inhibitor fragments capable of forming a covalent bond with the receptor; (f) ranking the electrophilic portion of potential covalent inhibitors or covalent inhibitor fragments for one or more characteristics such as Van der Waals radius, angle of attack, solvation effects, cone angles, electronic characteristics, bond length, bond strength, or combinations thereof; (g) and selecting the highly ranked electrophilic portion of the potential covalent inhibitors or covalent inhibitor fragments. In general, the highly ranked electrophilic portion of the covalent inhibitors or covalent inhibitor fragments may participate in a two-step mechanism consisting of: (1) a first reversible binding step leading to a non-covalent receptor-inhibitor adduct; and (2) then an irreversible binding to the receptor. In one embodiment, known, naturally occurring inhibitors may be used as the basis for making covalent inhibitors. Naturally occurring inhibitors have evolved to be biocompatible with biological systems and therefore should not cause non-specific covalent labeling of proteins.

The method to identify and select highly ranked electrophilic portion of the potential covalent inhibitors commences by identifying a potential receptor containing at least one nucleophilic group. The potential receptor can be any receptor when bound to a covalent drug provides a beneficial therapeutic effect. In one embodiment, the covalent drug does not cause any effect or causes a minimal adverse effect(s). Receptors, located on both the cell surface and within the cell, may be a protein, a nucleic acid, or a polypeptide. Non-limiting examples of proteins may be G protein-coupled receptors, enzymes (such as protein kinase, proteases, esterases, and phosphatases, ubiquitin ligases, deubiquitinating enzymes), ion channels, nuclear hormone receptors, structural proteins, chaperones, and membrane transport proteins.

The nucleophilic group within the receptor may be any nucleophilic group as part of a protein, a nucleic acid, or a polypeptide, or unnatural aminoacid. Non-limiting examples of nucleophilic groups may be sulfur atoms, nitrogen atoms, or oxygen atoms in an amino acid or nucleic acid. In one embodiment, the receptor was previously subjected to x-ray crystal analysis or other methods known in the art, and its three-dimensional structure is known.

The method of identifying and selecting a highly ranked electrophilic portion of the potential covalent inhibitors or covalent inhibitor fragments further comprises identifying known inhibitors or inhibitor fragments for a receptor. These known inhibitors comprise of an electrophilic or reactive groups that irreversibly binds to the receptor and form a chemical bond with the receptor and are generally located in a terminal position of the inhibitor. The known inhibitors or inhibitor fragments of the receptor may be derived from natural products, enzyme inhibitors, or drugs. Natural products, in the broadest sense, are those compounds derived from natural sources that contain an electrophilic group which produces a therapeutic response with a receptor. Non-limiting examples of sources of natural products may be bacteria, archaea, fungi, plants, and animals as well as humans. Non-limiting examples of enzyme inhibitors may be reversible or irreversible inhibitors. The inhibitors may function as an agonist, an inverse agonist, an antagonist, or a neutral antagonist. Drugs, in the broadest sense, are molecules other than food which can be inhaled, injected, smoked, consumed, absorbed through the skin, or dissolved under the tongue causes a physiological change in the body. Once bound to the receptor, the covalent inhibitors or covalent inhibitor fragments provide a beneficial therapeutic effect while minimizing any unwanted therapeutic effect(s). Some examples of suitable natural products are shown below:

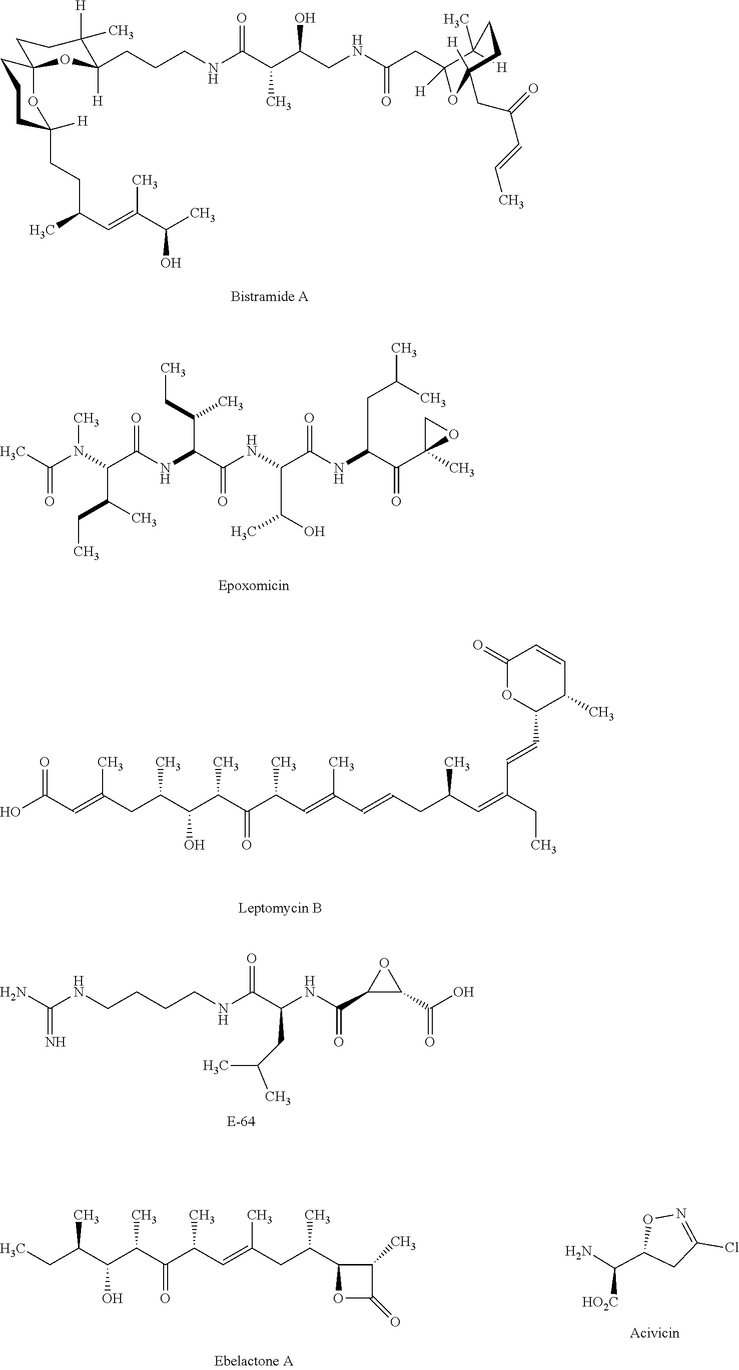

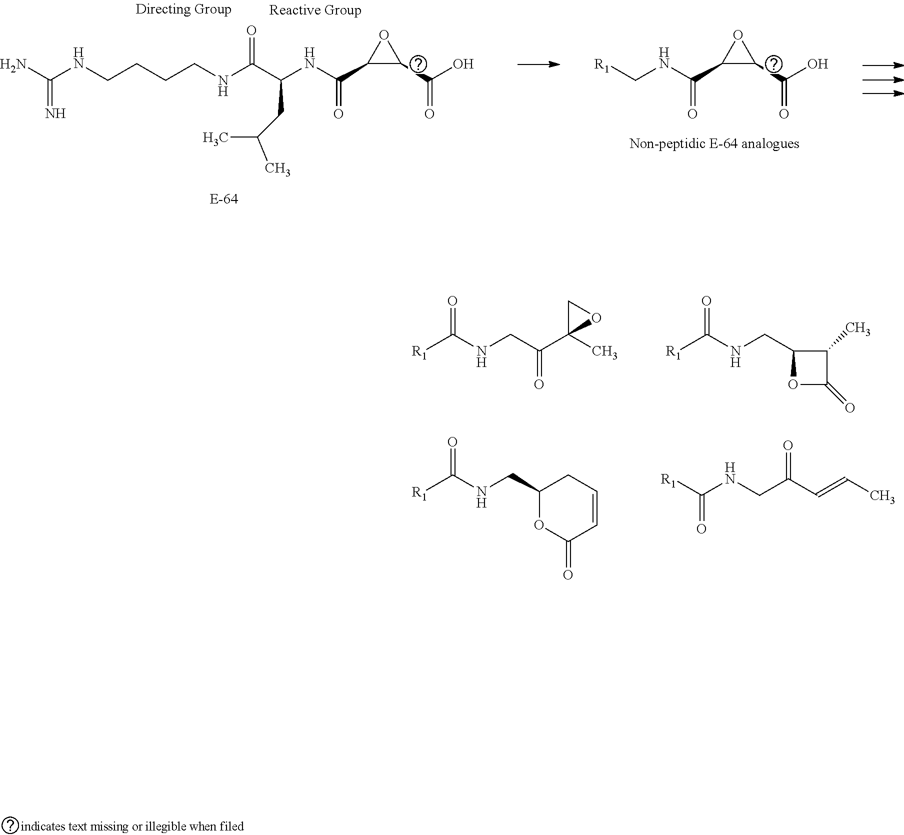

A known covalent drug or selective covalent probe is shown below:

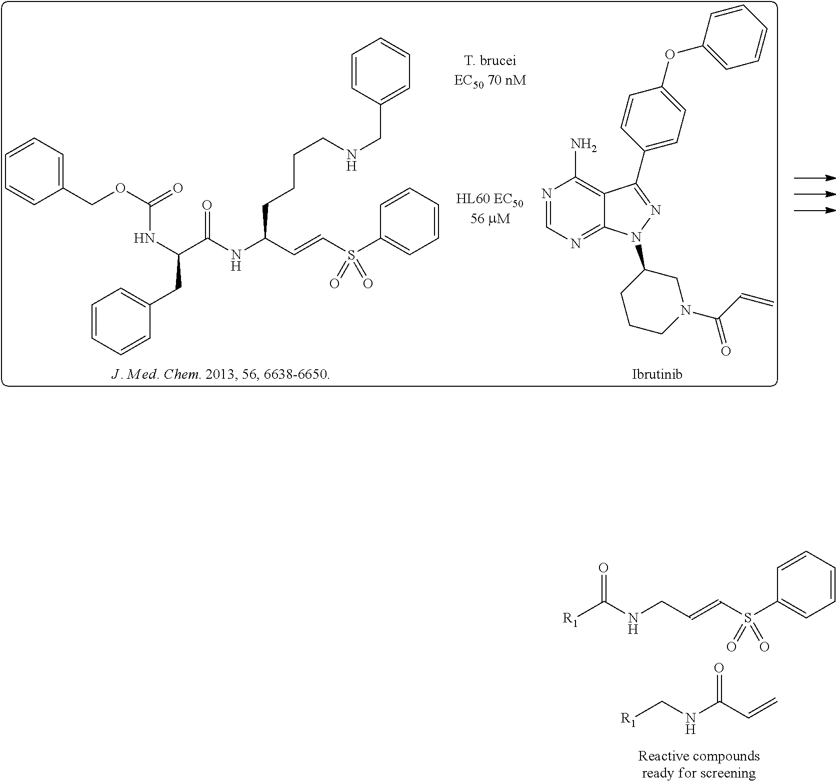

Generally, the reactive group may be any group which will bind to the nucleophilic center within the receptor. In various embodiments, the reactive group is an electrophilic group. In one example, the electrophilic group may be a Michael acceptor which may contain an electron donating, neutral, or electron withdrawing group. In other embodiments, the reactive group or electrophilic group may be a Michael acceptor containing an electron withdrawing group (EWG). Non-limiting examples of electron withdrawing groups may be nitros, amides, esters, acid, nitriles, ketones, sulfones, sulfoxides, sulfonamides, nitriles, halides, lactams, lactones, oxygen heterocycles, nitrogen heterocycles, substituted or unsubstituted aromatic fragments, and epoxides. Non-limiting examples of Michael acceptors that may be acceptors containing sp2 hybridized double and triple carbon-carbon bonds as well as epoxides attached to electron withdrawing group using carboxylic acid fragments are shown below:

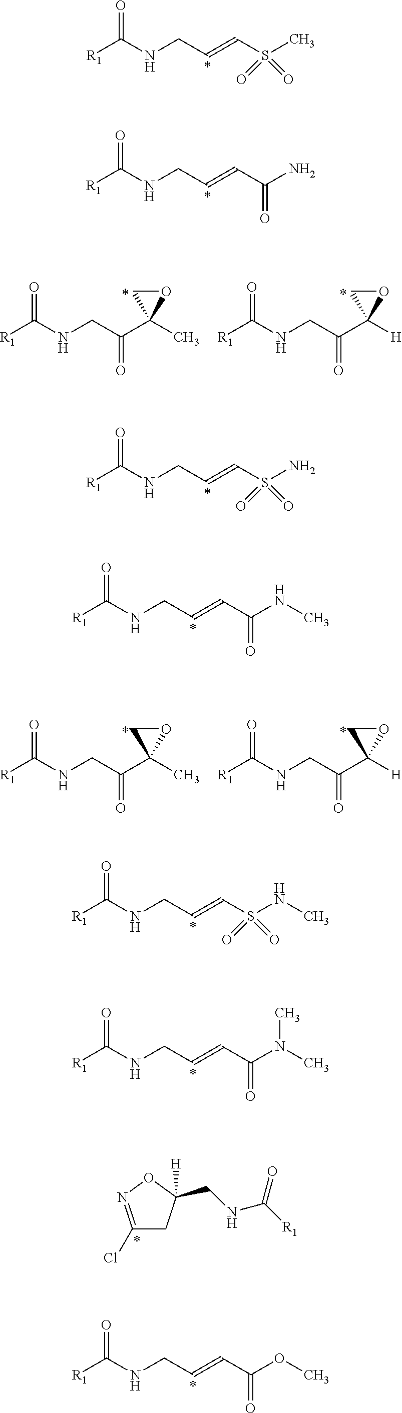

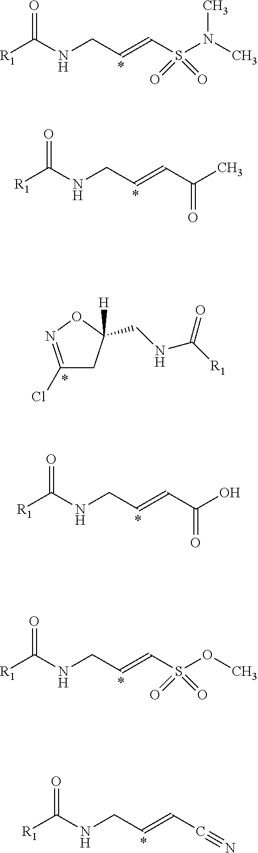

Non-limiting examples of Michael acceptors that may be acceptors containing sp hybridized double and triple carbon-carbon bonds attached to electron withdrawing group using carboxylic acid fragments are shown below:

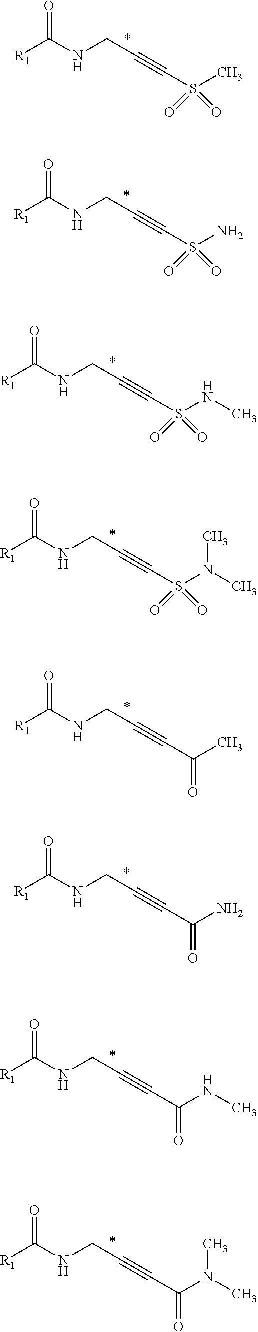

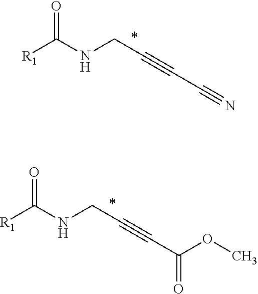

Non-limiting examples of electrophilic fragments based on amines are shown below:

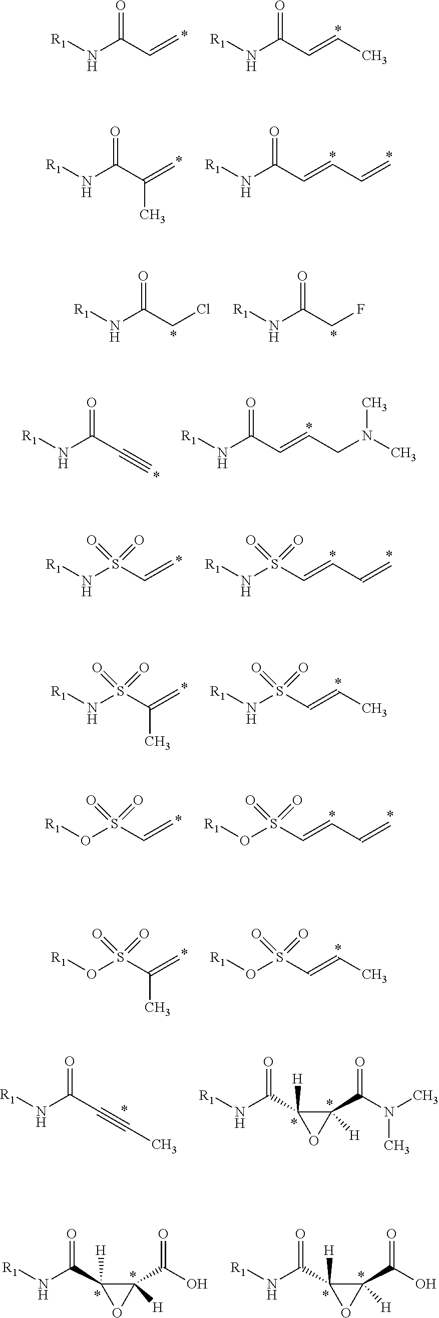

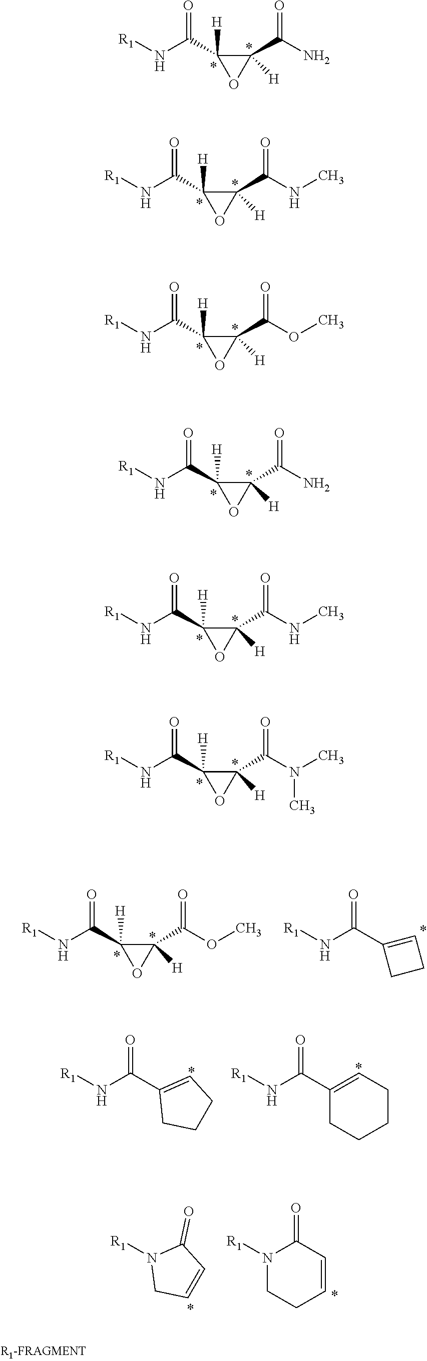

The known inhibitor or inhibitor fragment may be a natural product comprising a biosynthetically derived directing group and a reactive group which may bind to the receptor. In one embodiment, the reactive or electrophilic group is positioned at the terminal end of the natural product. The natural product may bind the receptor reversibly, and then may bind to the receptor irreversibly through a reactive group. In other embodiments, the known inhibitor or inhibitor fragment may be an enzyme inhibitor or a known drug.

Since the reactive group has been identified from a natural product, enzyme inhibitor, or drug which is biocompatible with the biological system, the reactive group should not cause non-specific binding with the receptor. Therefore, the reactive group should not cause hyperactive binding and produce no false positive hits.

The method to identify and select highly ranked electrophilic portion of the potential covalent inhibitors further comprises using a computer algorithm to dock the potential covalent inhibitor into a receptor. The algorithm used in the method identifies the biocompatible directing group and the reactive or electrophilic group within the natural product structure, enzyme inhibitor, or known drug. In some embodiments, the potential covalent inhibitor may be docked to a binding region of the receptor. In some embodiments, the binding region of the receptor is a ligand binding site. In some embodiments, the binding region of the receptor is a ligand binding pocket.

In some embodiments, the ligand-binding region of the receptor is known. In some embodiments, the ligand-binding region may be identified based on three-dimensional structure information of the receptor. The three-dimensional structure information may be obtained by X-ray crystallography, NMR spectroscopy, or cryo-electron microscopy (cryo-EM). In some embodiments, the three-dimensional structure information may be retrieved from a public structure database. The public structure database may include, for example, the Public Data Bank (PDB) (e.g., PDBe, PDBj, and RCSB).

Docking may be performed using a docking algorithm or program. Non-limiting examples of the docking algorithm or program include 1-Click Docking, AADS, ADAM, AutoDock, AutoDock Vina, BetaDock, Blaster, BSP-SLIM, CovDock, DARWIN, DIVALI, DOCK, DockingServer, Docking Study with HyperChem, DockVision, EADock, eHiTS, EUDOC, FDS, FlexX, FlexAID, FlexPepDock, FLIPDock, FLOG, FRED, FTDOCK, GEMDOCK, Glide, GOLD, GPCRautomodel, HADDOCK, Hammerhead, ICM-Dock, idTarget, iScreen, Lead finder, LigandFit, LigDockCSA, LIGIN, LPCCSU, MCDOCK, MEDock, Molecular Operating Environment(MOE), MolDock, MS-DOCK, ParDOCK, PatchDock, PLANTS, PLATINUM, PRODOCK, PSI-DOCK, PSO@AUTODOCK, PythDock, Q-Dock, QXP, rDock, SANDOCK, Score, SODOCK, SOFTDocking, Surflex-Dock, SwissDock, VoteDock, YUCCA, MOLS 2.0. In some embodiments, docking is performed using GLIDE. In some preferred embodiments, docking is performed using CovDock.

The term “docking” refers to the process of attempting to fit a three-dimensional configuration of a binding pair member into a three-dimensional configuration of the binding site or binding pocket of the partner binding pair member, which can be a protein, and determining the extent to which a fit is obtained. The extent to which a fit is obtained can depend on the amount of void volume in the resulting binding pair complex (or target molecule-ligand complex). The configuration can be physical or a representative configuration of the binding pair member, e.g., an in silico representation or other models.

The term “binding pocket” refers to a specific volume within a binding site. A binding pocket is a particular space within a binding site at least partially bounded by target molecule atoms. Thus a binding pocket is a particular shape, indentation, or cavity in the binding site. Binding pockets can contain particular chemical groups or structures that are important in the non-covalent binding of another molecule such as, for example, groups that contribute to ionic, hydrogen bonding, Van der Waals, or hydrophobic interactions between the molecules.

The term “binding site” refers to an area of a target molecule to which a ligand can bind non-covalently. Binding sites embody particular shapes and often contain multiple binding pockets present within the binding site. The particular shapes are often conserved within a class of molecules, such as a molecular family. Binding sites within a class also can contain conserved structures such as, for example, chemical moieties, the presence of a binding pocket, and/or an electrostatic charge at the binding site or some portion of the binding site, all of which can influence the shape of the binding site.

As used herein in connection with the design or development of ligands, the term “bind” and “binding” and like terms refer to a non-covalent energetically favorable association between the specified molecules (i.e., the bound state has a lower free energy than the separated state, which can be measured calorimetrically). For binding to a target, the binding is at least selective, that is, the compound binds preferentially to a particular target or members of a target family at a binding site, as compared to non-specific binding to unrelated proteins not having a similar binding site. For example, BSA is often used for evaluating or controlling for non-specific binding. In addition, for an association to be regarded as binding, the decrease in free energy going from a separated state to the bound state must be sufficient so that the association is detectable in a biochemical assay suitable for the molecules involved.

The molecular parameters that govern binding of substrates and inhibitors to enzymes are well known in the art. Typically, binding is governed by hydrogen bonding, hydrophobic interactions, ionic bonds (salt links), covalent bonds (at certain stages of the reaction), and Van der Waals forces; binding typically involves either a “lock and key” mechanism or an “induced fit” mechanism. These can be modeled by means of appropriate software, taking into account the variation in the strength of the interaction with the distance between the two molecules and that there are six degrees of rotational and translational freedom of one molecule relative to the other as well as the conformational degrees of freedom of each molecule.

The method to identify and select highly ranked electrophilic portion of the potential covalent inhibitors further comprises analyzing the nucleophilic character of the receptor to electrophilic groups of potential covalent inhibitor. The algorithm may analyze the nucleophilic character of the receptor and matches this nucleophilic character to the reactive group or the electrophilic group of the potential covalent inhibitor or inhibitor fragment. In this analysis, the algorithm measures one or more of the following characteristics such as Van der Waals radius, bond length, bond strength, angle of attack, cone angle, electronic character, or a combination thereof. One or more of these characteristics will be used to match the appropriate reactive or electrophilic group with the nucleophilic site within the receptor.

The method further comprises identifying the electrophilic portion of the potential covalent inhibitors capable of forming a covalent bond with the receptor. The algorithm, using the data from the previous step, identifies the electrophilic portion of the potential covalent inhibitor capable of forming a covalent bond with the nucleophilic portion of the inhibitor based on the characteristic determined above and determines the ability of the groups to form a covalent bond.

The method additionally comprises ranking the electrophilic portion of the potential covalent inhibitors by one or more of the characteristics described above. This ranking will aid in the selection of candidates and provide covalent drugs which are not or are minimally hyperreactive.

The final step in the method comprises selecting the highly ranked candidates of the electrophilic portion of the potential covalent inhibitors or covalent fragments. In various embodiments, the selection may be based on one or more of the characters described above. The ranking may also depend on the availability of commercial sources of the natural product, enzyme inhibitors, or known drugs. The potential covalent inhibitor may be used as an agonist, an inverse agonist, an antagonist, or a neutral antagonist.

In another aspect, the method further comprises preparing highly ranked candidates of potential covalent inhibitors with a highly active electrophilic group for a screening library. This method, using the highly ranked electrophilic candidates identified above, comprises selecting the appropriate natural product candidate, enzyme inhibitor, or known drug candidate with the desired electrophilic or reactive group, cutting the bio synthetically derived directing group, and replacing the directing group form the drug or enzyme inhibitor with an amine or carboxylic acid, and then coupling the amine or carboxylic acid moiety with R1-COOH or R₁—CH₂—NH₂ to produce an amide. In general, this method produces covalent candidates for a screening library by a robust method without using protecting groups.

Once the candidates are prepared for a screening library, the method further comprises experimentally evaluating the candidates through assays. These assays may provide data regarding the potency and effectiveness of the covalent inhibitor. In addition, the method further comprises measuring the reaction rate of candidates comprising the same electrophile with glutathione, cysteine, or other nucleophiles. This data can further elucidate the potency and effectiveness of the candidates within a library, eliminate hyperreactive compounds, and can be utilized in a structure-activity relationship (SAR) study.

The term “assay” refers to experimental setups used to gather data regarding a particular result of the experimental conditions. For example, enzymes can be assayed based on their ability to act upon a detectable substrate. Likewise, for example, a compound or ligand can be assayed based on its binding affinity or inhibitory activity to bind to a particular target.

The method commences by selecting the appropriate natural product, enzyme inhibitor, or drug candidate with the desired electrophilic (reactive) group, cutting the biocompatible directing group, replacing the biocompatible directing group with a carboxylic acid or an amine, and then preparing the covalent inhibitor fragment by coupling the amine or carboxylic acid moiety with R₁—COOH or R₁—CH₂—NH₂ using a peptide coupling reaction to produce an amide. The peptide coupling reaction does not use protecting groups.

The synthetic preparation of the covalent inhibitor commences with the selection of the appropriate natural product, enzyme inhibitor, or known drug with the desired electrophilic group as determined by the above algorithm.

The next step in the preparation of the covalent inhibitor candidate is to separate the directing group from the biocompatible electrophilic group, accompanied by the installation of the amine/carboxylic acid group at the breaking point in the biocompatible electrophilic group. The resulting biochompatible electorphilic fragment can be coupled with the R₁—NH2/COOH (R₁ is a fragment) producing libraries of electrophilic fragments containing biocompatible electrophile and fragment connected via amide bond. In such method, the directing group in the original natural product is replaced with the drug-like fragment R₁ group. Cleavage and the replacement of the biocompatible directing group may be accomplished using common chemistry knowledge, as to (a) preserve chemical reactivity of the reactive group, (b) generate a chemically stable compound that can be prepared using known literature methods, or their modifications, and (c) ensure minimal linker (1-2 atoms) between the amine/carboxylic group and the biocompatible reactive group. If the resulting compound is not chemically stable as assessed by using common chemistry knowledge, one carbon should be inserted between the amine/carboxylic acid and the reactive group. In some embodiments, this cleavage of the biocompatible directing group yields either an amine or a carboxylic acid. In other embodiments, the amine or carboxylic acid would be chemically inserted into the electrophilic fragment. In either method, the reactive or electrophilic group comprises an amine or a carboxylic acid and amide bonds derived from amines or carboxylic acids.

The final step in preparing the covalent inhibitor candidate is to couple the electrophilic fragment, comprising either an amine or a carboxylic acid with either R₁—COOH or R₁—CH₂—NH₂ to form an amide bond wherein R₁ is a drug-like molecule or a fragment comprising 10-17 nonhydrogen atoms without using protecting groups. Methods for forming an amide or a peptide bond are known to the skilled artisan. Non-limiting examples of peptide coupling reagents may be N,N′-dicyclohexylcarbodiimide, N,N′-diisopropylcarbodiimide, HATU (1-[bis(dimethylamino) methylene]-1H-1,2,3-triazolo[4,5-b]pyridinium 3-oxide hexafluorophosphate), HBTU (2-(1H-benzotriazol-1-yl)-1,1,3,3-tetramethyluronium hexafluorophosphate), HOAt (1-hydroxy-7-aza-benzotriazole), HOBt (hydroxybenzotriazole), PyAOP (7-azabenzotriazol-1-yloxy) tripyrrolidino-phosphonium hexafluorophosphate), PyBOP (benzotriazol-1-yl-oxytripyrrolidino-phosphonium hexafluorophosphate), or combinations thereof. In various embodiments, the coupling may be performed in the presence of a base. Non-limiting examples of a base may be an organic base such as triethylamine, N,N-diisopropylethylamine, N-methyl morpholine, or an inorganic base such as sodium hydroxide, potassium hydroxide, sodium bicarbonate. A general method of linking the electrophilic group and the directing group through a peptide coupling is shown below:

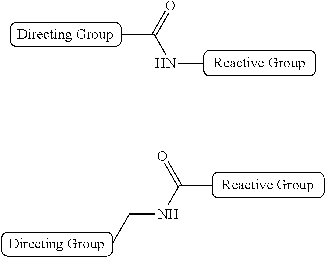

The design and synthesis of a fragment library using a representative electrophilic (reactive) group is illustrated below:

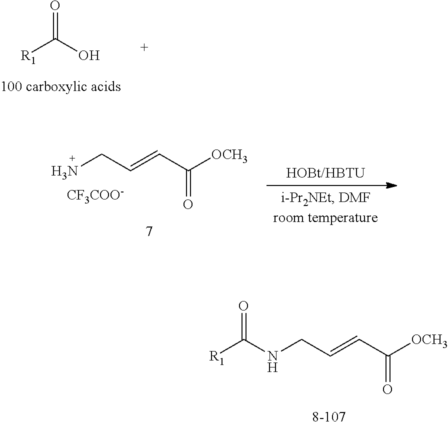

Examples of representative covalent inhibitors generated from the method are shown below:

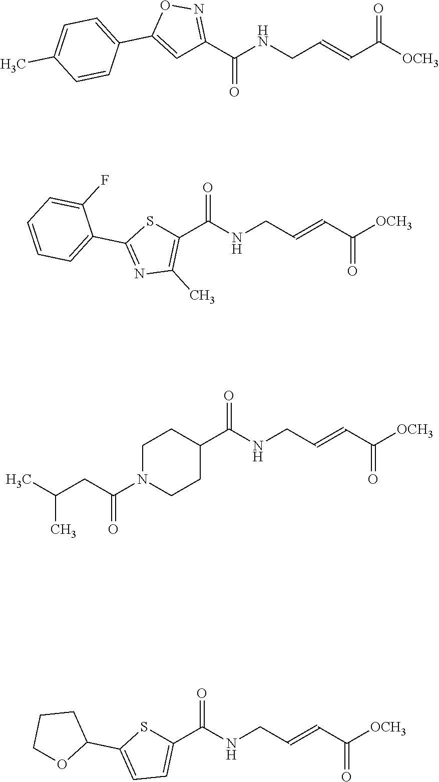 [SMILES: COC(=O)/C=C/CNC(=O)c1cc(-c2ccc(C)cc2)on1 ; COC(=O)/C=C/CNC(=O)c1sc(-c2ccccc2F)nc1C ; COC(=O)/C=C/CNC(=O)C1CCN(C(=O)CC(C)C)CC1 ; COC(=O)/C=C/CNC(=O)c1ccc(C2CCCO2)s1]

In a further embodiment, once the potential covalent inhibitor candidate libraries have been prepared, these candidates can be evaluated by dosing them in a specific assay at a specific concentration versus the receptor to determine the potency of each of the candidates. Depending on the covalent inhibitor, the receptor, and the therapeutic area, the assays will vary. Methods of performing wet chemistry assays are well known. Examples of covalent fragments evaluated in the enzyme assays against cysteine protease papain is shown below (J Med Chem. 2014 Jun. 12; 57(11):4969-74):

| Known inhibitors of papain, k_{inact}/Kᵢ, M⁻¹ s⁻¹ | | Inhibitors of papain in this work, k_{inact}/Kᵢ, M⁻¹ s⁻¹ | |
|---|---|---|---|
| 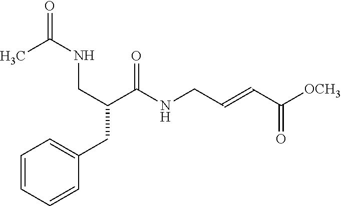 [SMILES: COC(=O)/C=C/CNC(=O)[C@@H](CNC(C)=O)Cc1ccccc1] | 6.575 | 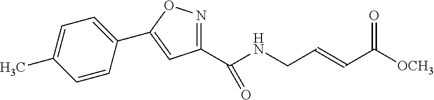 [SMILES: COC(=O)/C=C/CNC(=O)c1cc(-c2ccc(C)cc2)on1] | 1.228 |
| 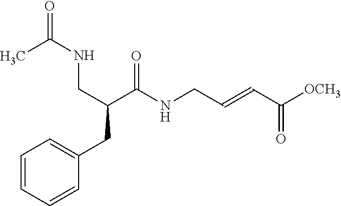 [SMILES: COC(=O)/C=C/CNC(=O)[C@H](CNC(C)=O)Cc1ccccc1] | 0.386 | 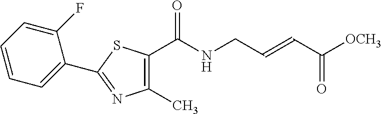 [SMILES: COC(=O)/C=C/CNC(=O)c1sc(-c2ccccc2F)nc1C] | 0.409 |
| 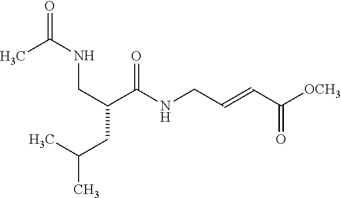 [SMILES: COC(=O)/C=C/CNC(=O)[C@@H](CNC(C)=O)CC(C)C] | 1.102 | 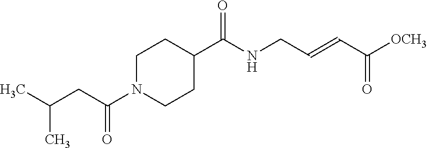 [SMILES: COC(=O)/C=C/CNC(=O)C1CCN(C(=O)CC(C)C)CC1] | 0.461 |
| | | 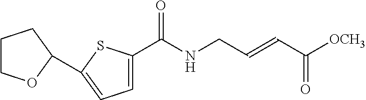 [SMILES: COC(=O)/C=C/CNC(=O)c1ccc(C2CCCO2)s1] | 0.037 |

The library of potential covalent inhibitors comprising the same electrophilic groups may be further evaluated by measuring the reaction rate. This method consists of dosing potential covalent inhibitor with the same electrophilic group within a library with a model of the receptor containing a nucleophilic group, then measuring the reaction rate by an analytical method. This measurement would provide additional data and an assay to prepare more highly potent covalent drugs.

As previously indicated, the nucleophilic group within the receptor may be part of a protein, a nucleic acid, or a polypeptide. Models of the nucleophilic group within the receptor may be an amino acid or an amino acid comprising a protecting group. Non-limiting examples of the nucleophilic group within a receptor may be any known amino acid. Preferred amino acids which contain a nucleophilic group may be cysteine. In various embodiments, the cysteine may be functionalized to protect the carboxylic acid as an ester and to protect the nitrogen as an amide. Analytical techniques may be employed to determine the reaction rate. Non-limiting examples of analytical techniques may be HPLC, GC, and NMR. A preferred analytical technique may be NMR. Evaluation of the data would then provide the relative reaction rate and be used in a SAR study and eliminate potentially hyperreactive compounds. This rate data would then be used to determine the appropriate candidate.

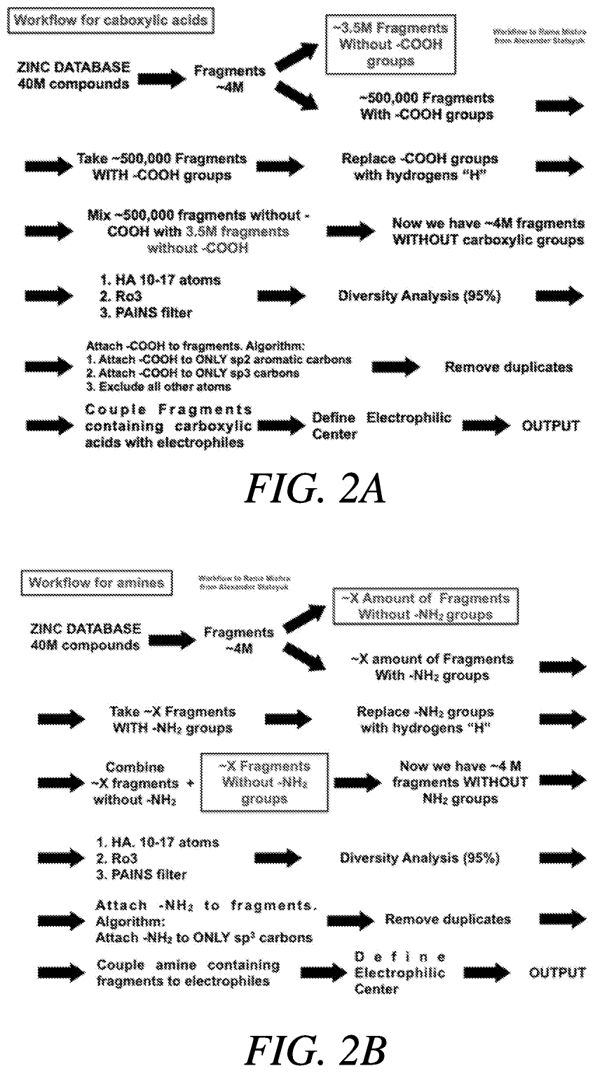

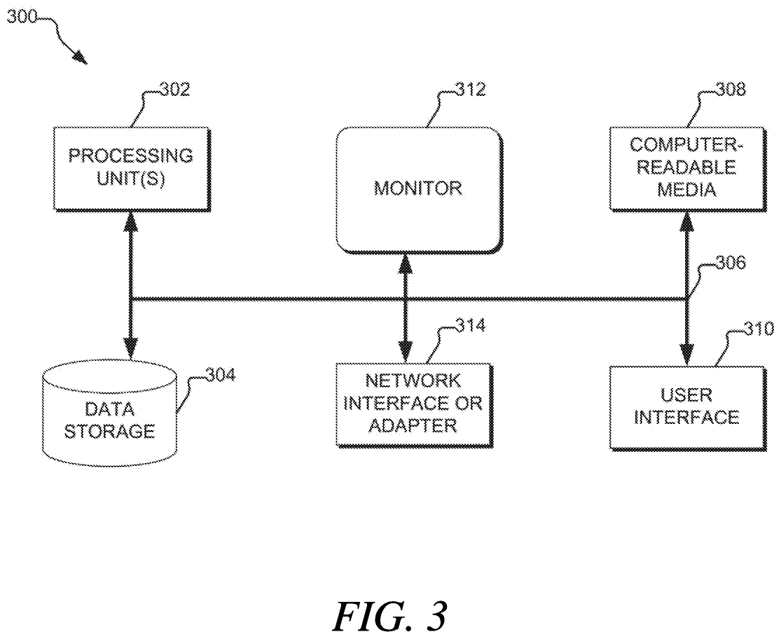

The methods, as detailed above, for using the computer algorithm to create libraries of covalent fragments for virtual docking libraries and synthesizing the libraries has been detailed in FIG. 3. FIGS. 2A-2B show the methods applied to a known database. Examples of the database may include, for example, ZINC, ChEMBL, NCI, PubChem, ChemDB, and BindingDB. The ZINC database which contains more than 40 million compounds. FIG. 2A shows the above-described methods applied to this database, specifically a general workflow for carboxylic acids. FIG. 2B details the same methods applied to a workflow for amines.

FIG. 3 illustrates an example of a suitable computing and networking environment 300 that may be used to implement various aspects of the present disclosure, as well as the computing components of FIG. 1, such as the server device 102. As illustrated, the computing and networking environment 300 includes a general purpose computing device 300, although it is contemplated that the networking environment 300 may include one or more other computing systems, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments that include any of the above computing systems or devices, and the like.

Components of the computer 300 may include various hardware components, such as a processing unit 302, a data storage 304 (e.g., a system memory), and a system bus 306 that couples various system components of the computer 300 to the processing unit 302. The system bus 306 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computer 300 may further include a variety of computer-readable media 308 that includes removable/non-removable media and volatile/nonvolatile media, but excludes transitory propagated signals. Computer-readable media 308 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computer 300. Communication media includes computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The data storage or system memory 304 includes computer storage media in the form of volatile/nonvolatile memory such as read-only memory (ROM) and random access memory (RAM). A basic input/output system (BIOS), containing the basic routines that help to transfer information between elements within the computer 300 (e.g., during start-up) is typically stored in ROM. RAM typically contains data and/or program modules that are immediately accessible to and/or presently being operated on by processing unit 302. For example, in one embodiment, data storage 304 holds an operating system, application programs, and other program modules and program data.

Data storage 304 may also include other removable/non-removable, volatile/nonvolatile computer storage media. For example, data storage 304 may be: a hard disk drive that reads from or writes to non-removable, nonvolatile magnetic media; a magnetic disk drive that reads from or writes to a removable, nonvolatile magnetic disk; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media, described above and illustrated in FIG. 3, provide storage of computer-readable instructions, data structures, program modules and other data for the computer 300.

A user may enter commands and information through a user interface 310 or other input devices such as a tablet, electronic digitizer, a microphone, keyboard, and/or pointing device, commonly referred to as mouse, trackball or touch pad. Other input devices may include a joystick, game pad, satellite dish, scanner, or the like. Additionally, voice inputs, gesture inputs (e.g., via hands or fingers), or other natural user interfaces may also be used with the appropriate input devices, such as a microphone, camera, tablet, touch pad, glove, or other sensor. These and other input devices are often connected to the processing unit 302 through a user interface 310 that is coupled to the system bus 306 but may be connected by other interface and bus structures, such as a parallel port, game port or a universal serial bus (USB). A monitor 312 or other type of display device is also connected to the system bus 306 via an interface, such as a video interface. The monitor 312 may also be integrated with a touch-screen panel or the like.

The computer 300 may operate in a networked or cloud-computing environment using logical connections of a network interface or adapter 314 to one or more remote devices, such as a remote computer. The remote computer may be a personal computer, a server, a router, a network PC, a peer device or other common network node, and typically includes many or all of the elements described above relative to the computer 300. The logical connections depicted in FIG. 3 include one or more local area networks (LAN) and one or more wide area networks (WAN), but may also include other networks. Such networking environments are commonplace in offices, enterprise-wide computer networks, intranets and the Internet.

When used in a networked or cloud-computing environment, the computer 300 may be connected to a public and/or private network through the network interface or adapter 314. In such embodiments, a modem or other means for establishing communications over the network is connected to the system bus 306 via the network interface or adapter 314 or other appropriate mechanism. A wireless networking component including an interface and antenna may be coupled through a suitable device such as an access point or peer computer to a network. In a networked environment, program modules depicted relative to the computer 300, or portions thereof, may be stored in the remote memory storage device.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the disclosure and are thus within the spirit and scope of the present disclosure. From the above description and drawings, it will be understood by those of ordinary skill in the art that the particular embodiments shown and described are for purposes of illustrations only and are not intended to limit the scope of the present disclosure. References to details of particular embodiments are not intended to limit the scope of the disclosure.

### EXAMPLES

The following examples illustrate various embodiments of the aspects of the present application.

Examples for creating covalent inhibitor libraries from natural products

### Example 1

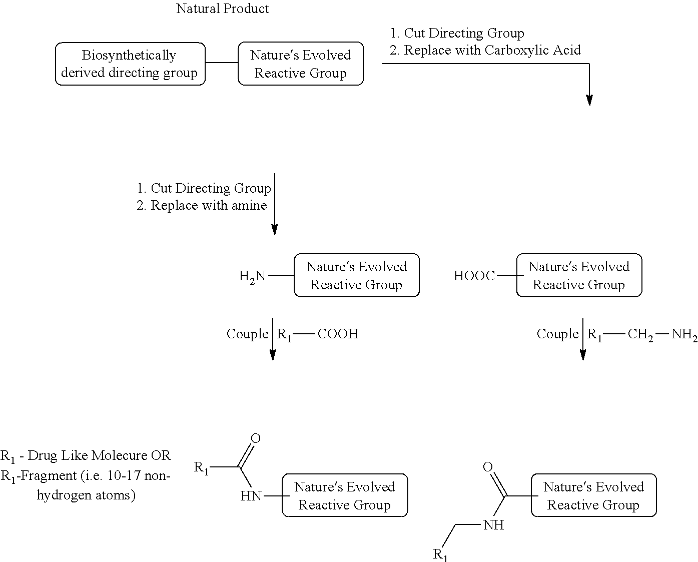

### Reactive Compound Ready for Screening

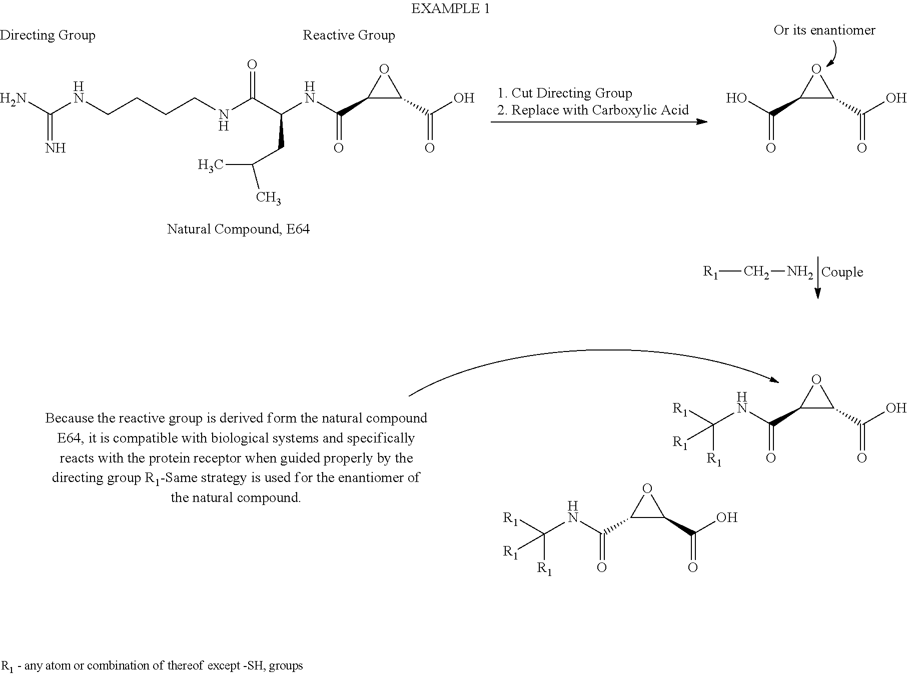

### Example 2

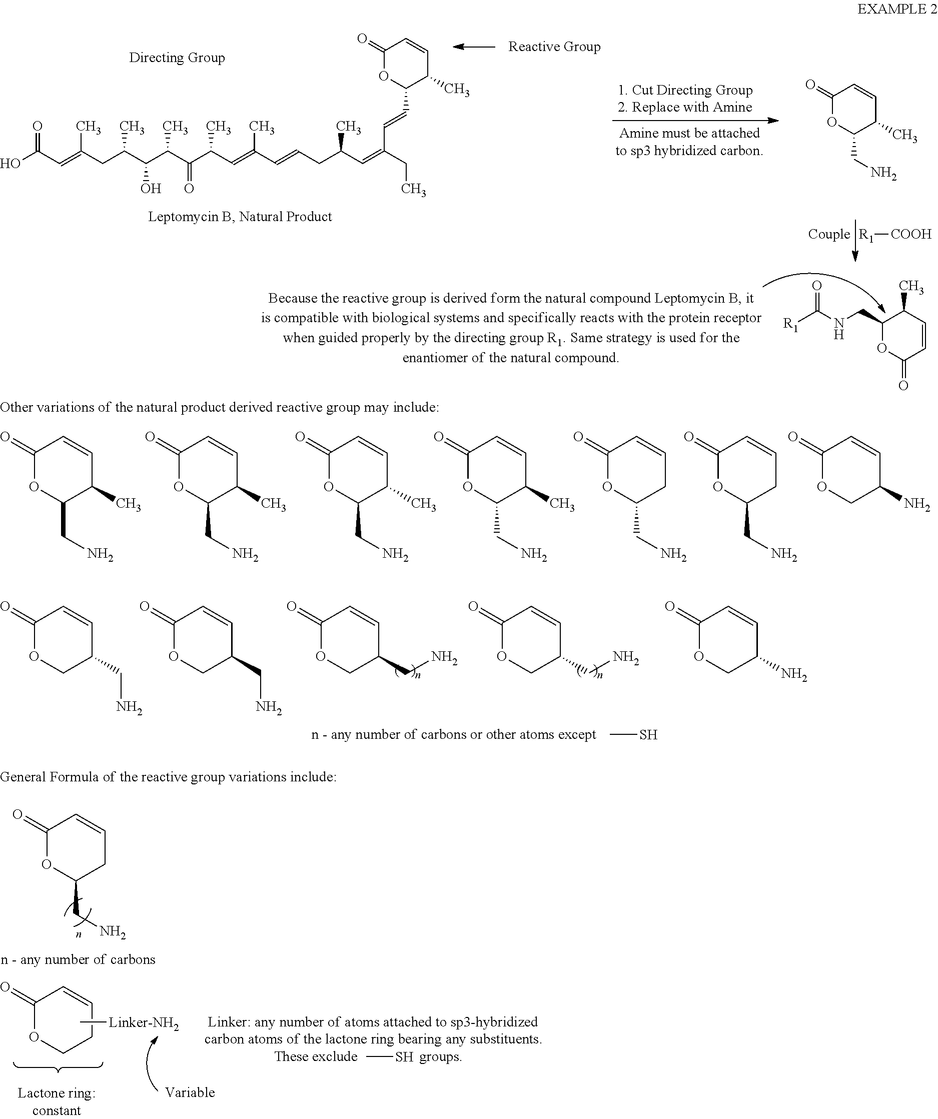

### Example 3

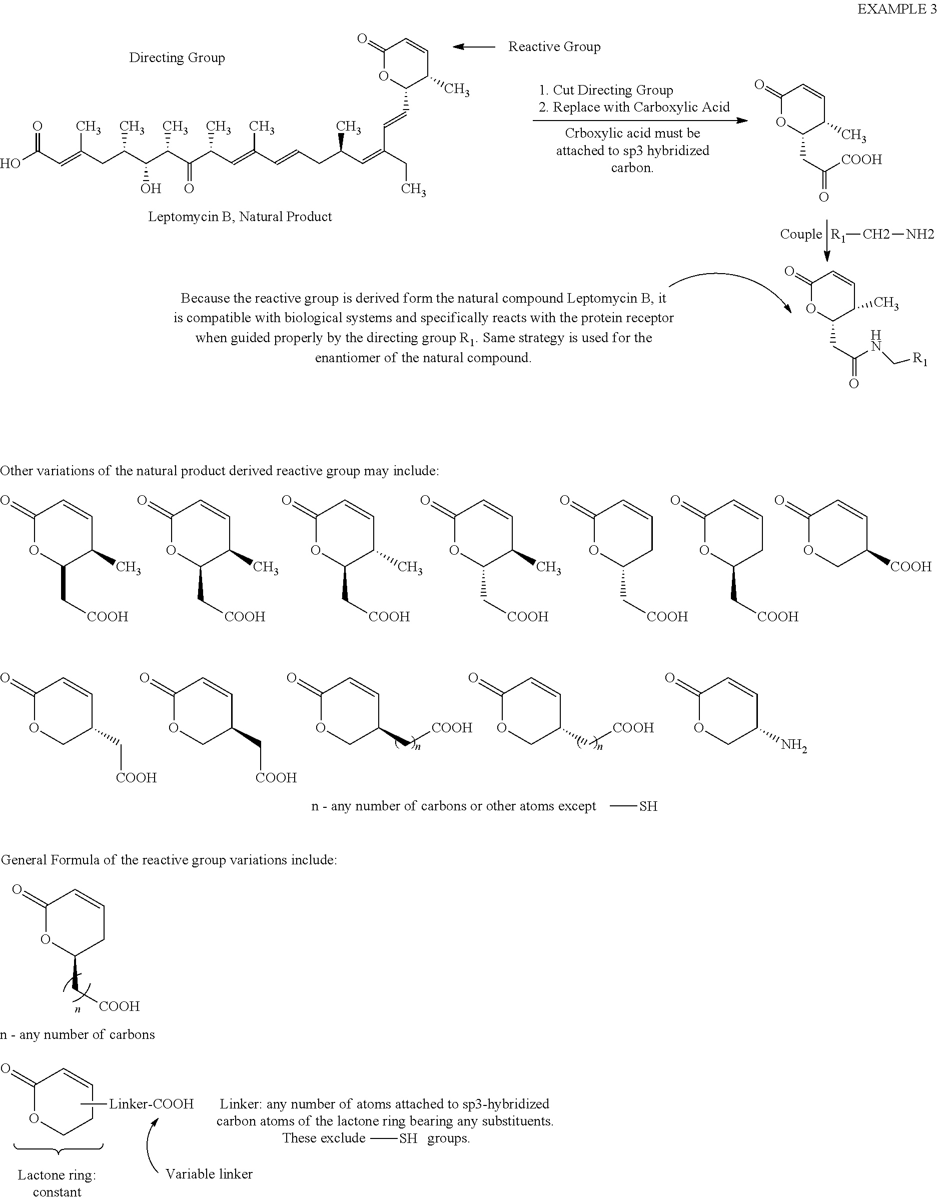

### Example 4

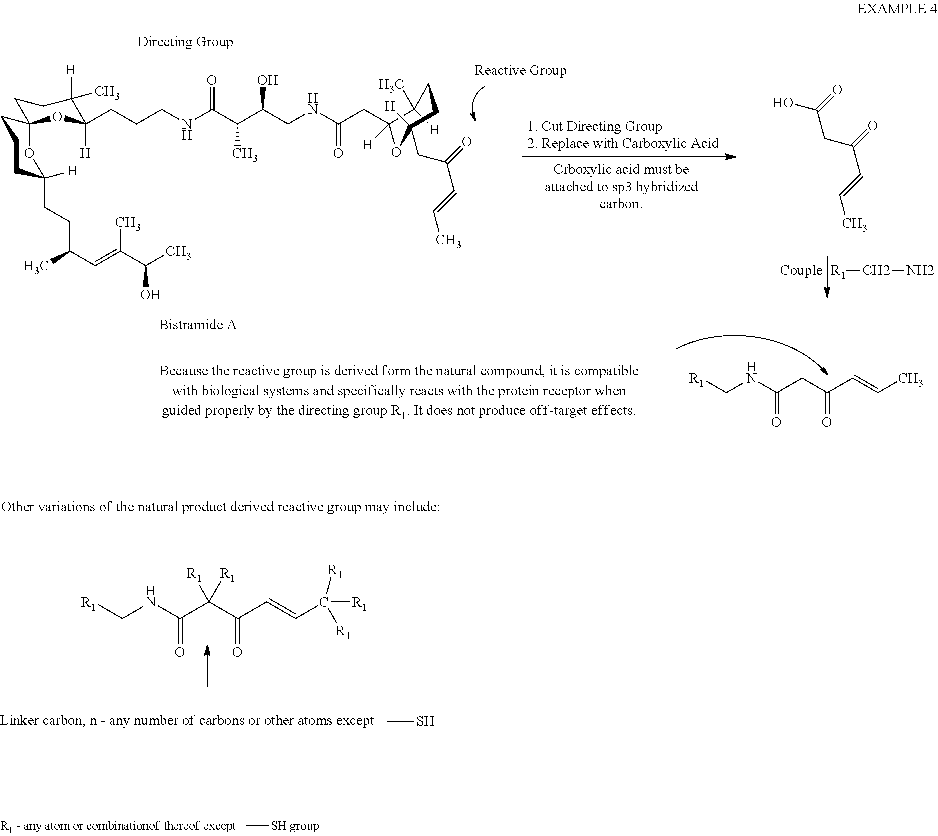

### Example 5

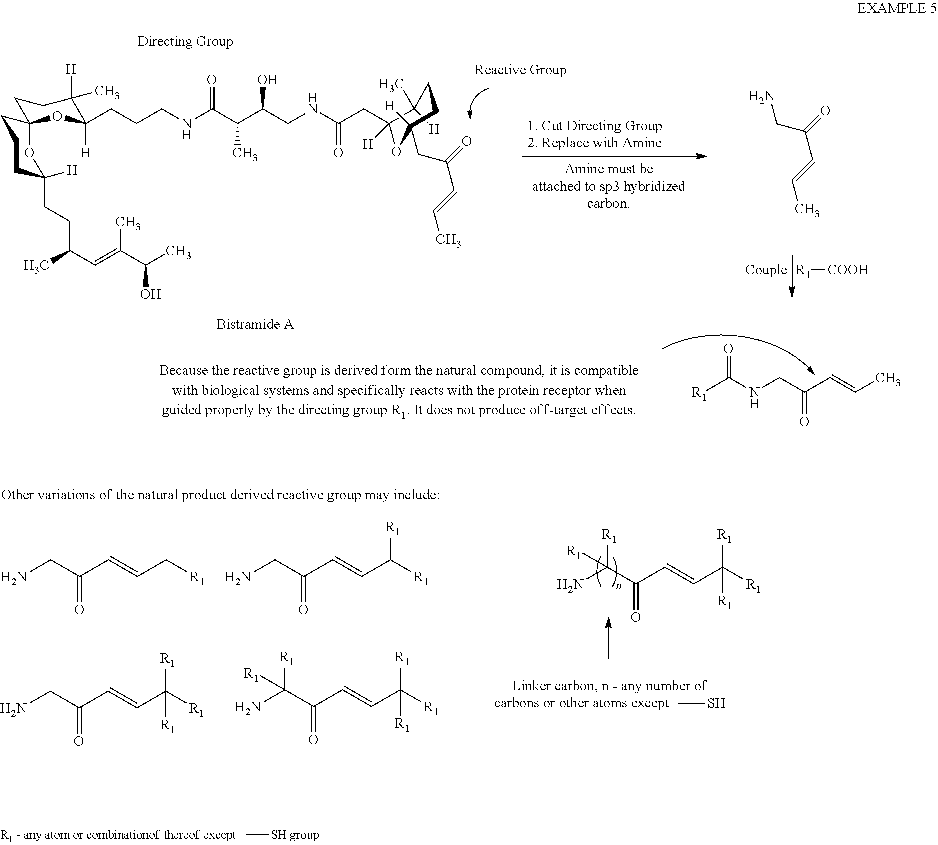

### Example 6

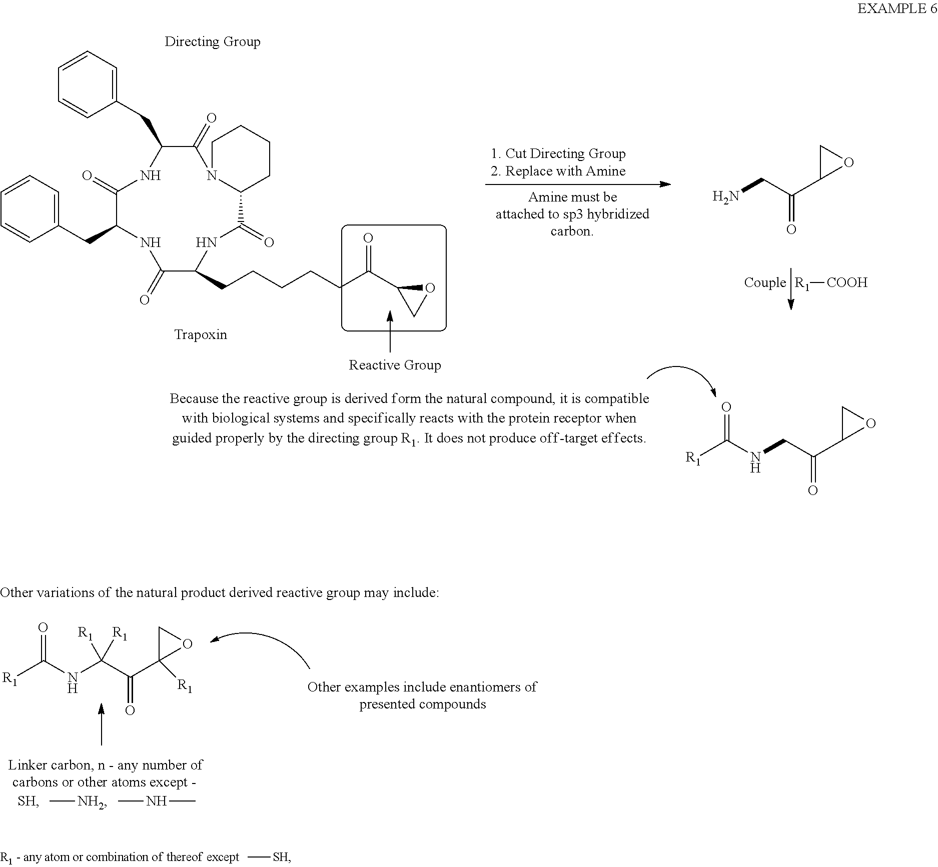

### Example 7

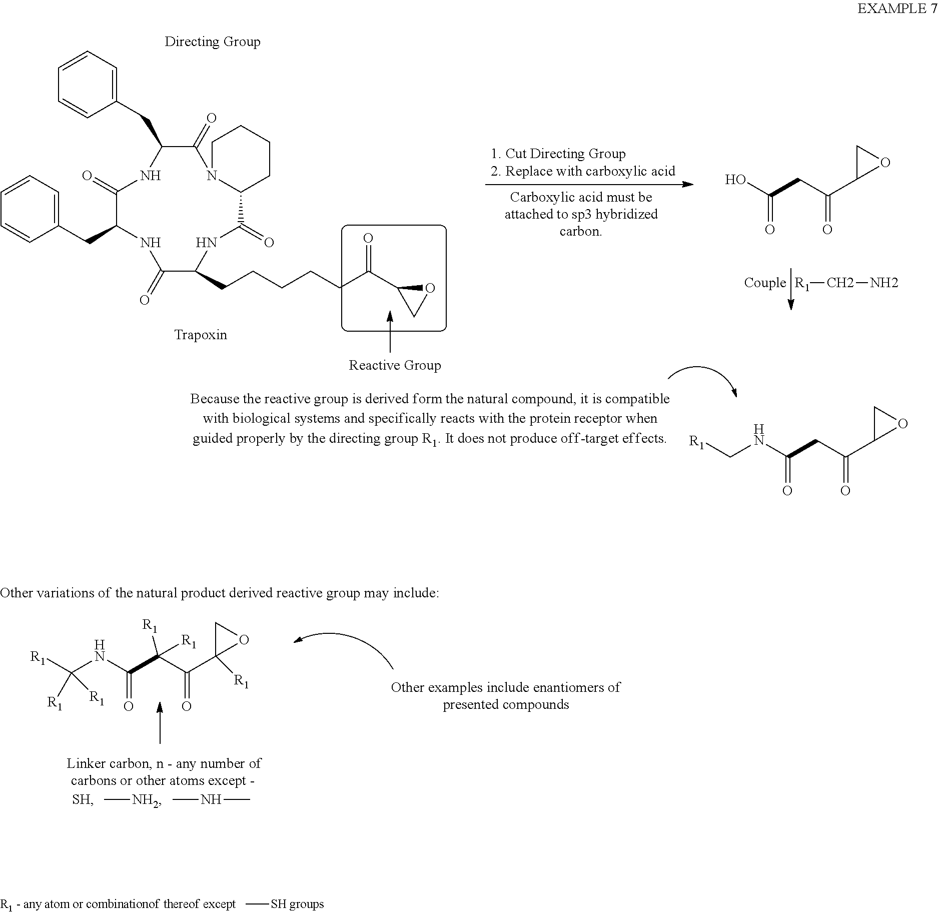

### Example 8

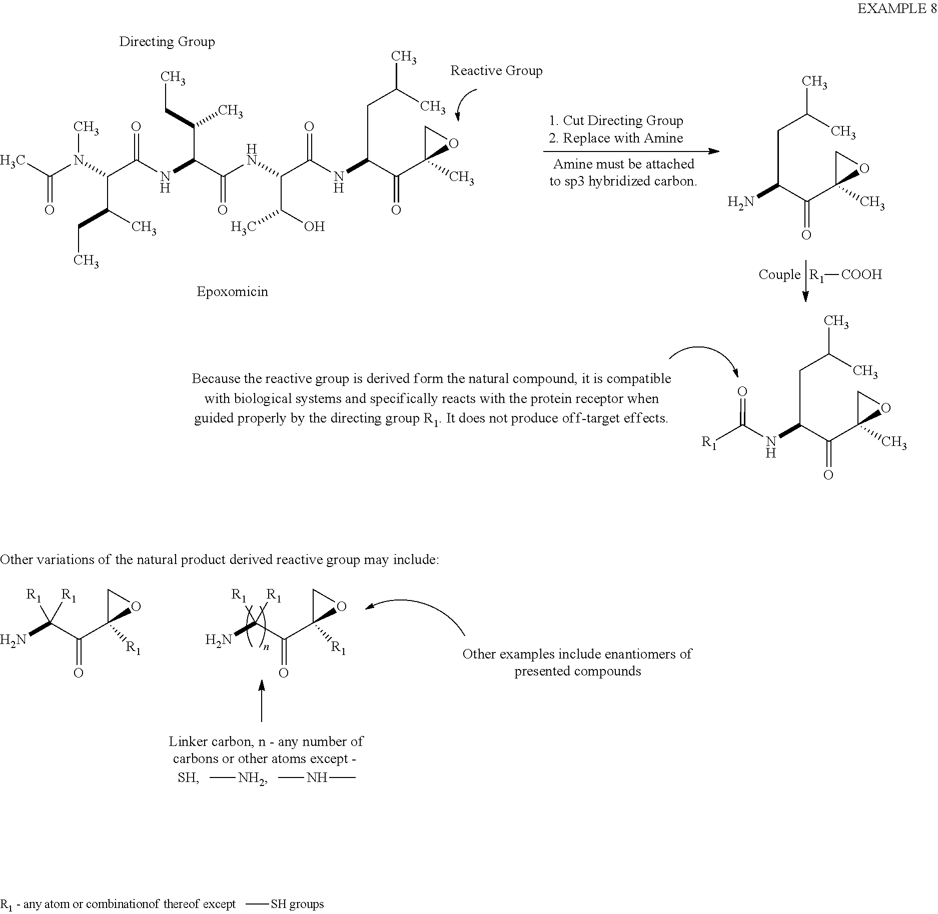

### Example 9

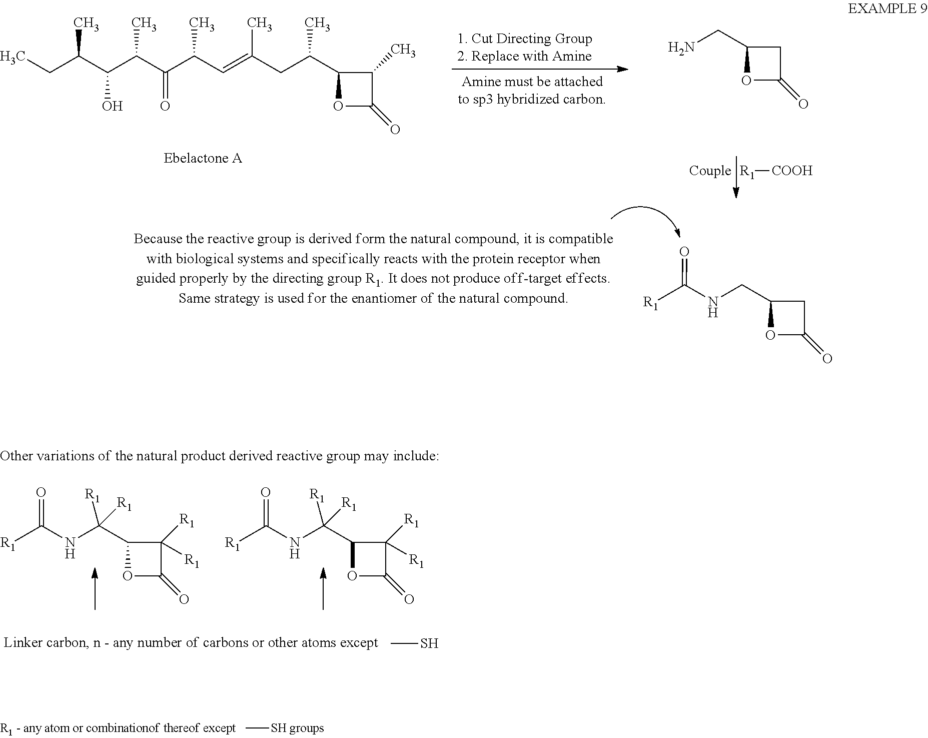

### Example 10

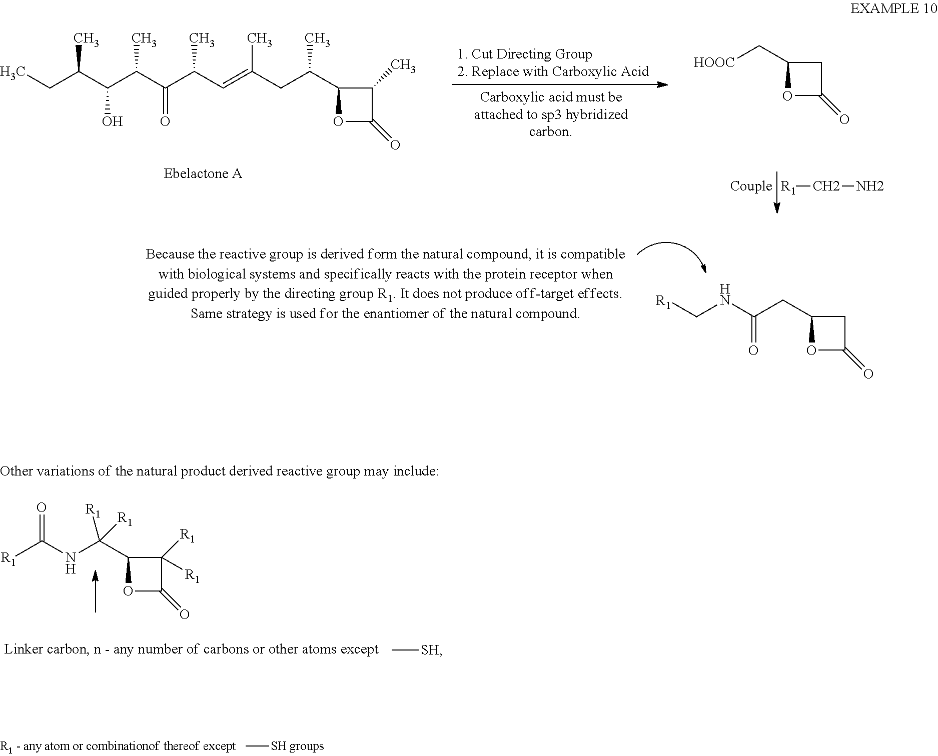

### Example 11

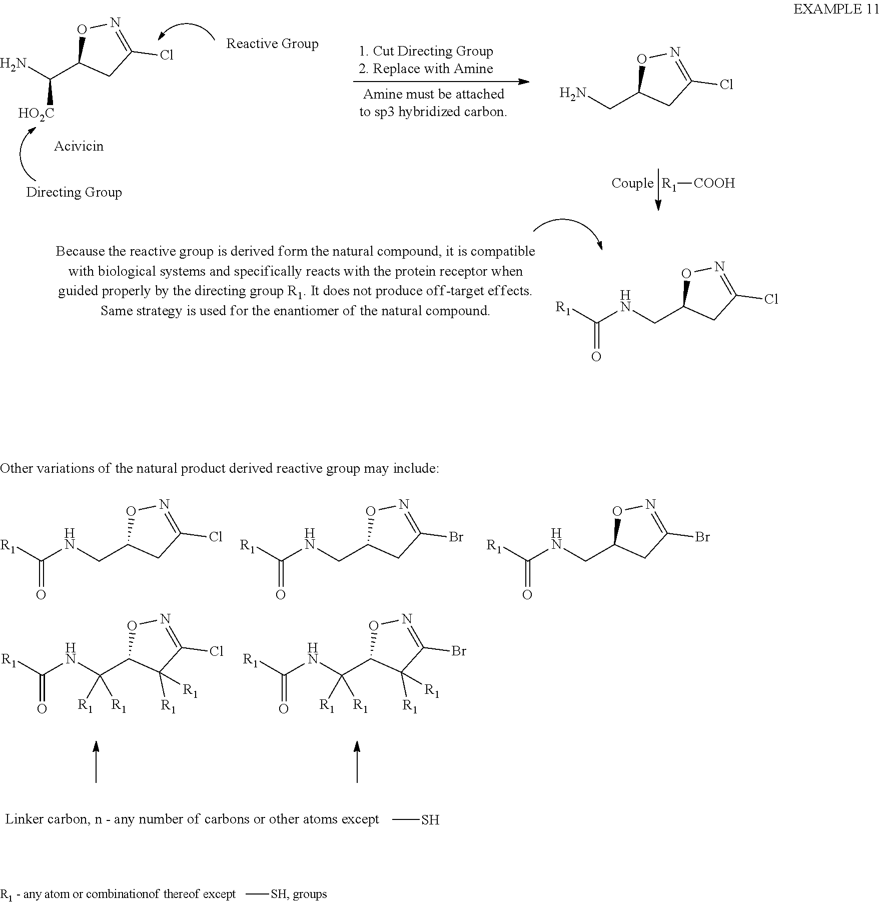

Examples of virtual docking of covalent fragment libraries prepared using present methods using Schrodinger CovDock performed at Northwestern University and experimental validation of identified hit compounds performed at the University of Houston.

### Example 12

### Construction of a Small Pilot Library of Covalent Fragments Using Developed Computer Algorithm and Commercially Available Carboxylic Acids

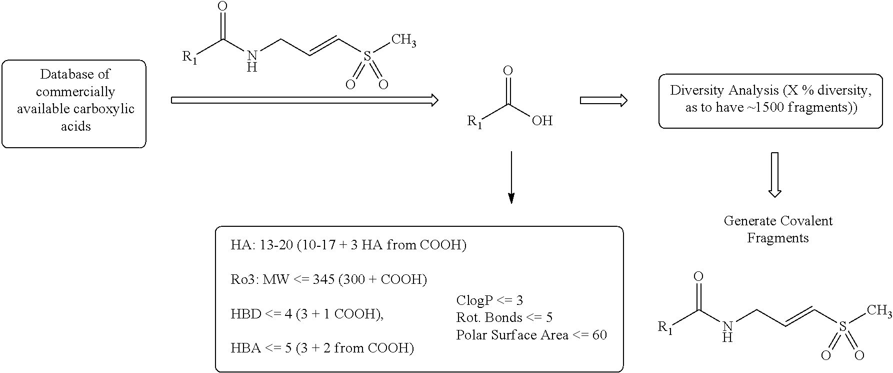

### Example 12

### Procedure for the Ccovalent Docking of the Resulting Vinyl Sulfone Fragments

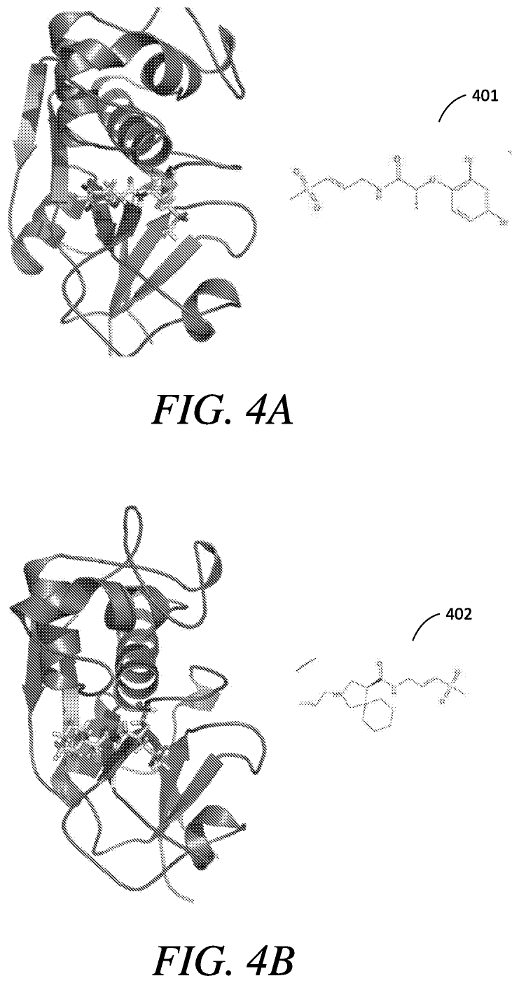

To carry out the covalent docking of the proposed ligands, the docking modules available in Schrodinger platform was used. These covalent docking tools require that the ligand set must be a series of related compounds and all of which should react with the receptor at the same site and by the same mechanism. The first step is the regular non-covalent docking of the ligands having the potential to form a covalent bond with the reactive residue of the receptor. Then the program allows for the different conformations of the side chain of the reactive residue, which is temporarily mutated to alanine at this stage. The reason for this temporary mutation is only to avoid the bias towards the particular ligand conformation if the side chain of the reactive residue is present. After the non-covalent docking, the original side chain of the reactive residue is replaced, and the covalent bond is formed. The ligands where the covalent bond lengths between the reactive center of the ligand and the reactive residue of the receptor are longer compared to the standard chemical bonds are discarded. After the bond formation between the potential covalent ligand and the receptor, the complex is minimized using the Prime module of Schrodinger suite. Finally, the docked poses of the covalently linked ligands are to be visualized and rank ordered by energy and the docked score. To dock these 1648 ligands, a cysteine protease of papain family, the human cathepsin L (hCAT-L) crystal structure (PDB ID: 4AXL) having 1.92 Å resolution was used. In the active site of this structure, Cys25 reactive residue is present. The reaction site of the ligands were determined using SMART search pattern implemented in the covalent docking program. After determining the reactive sites from both the ligands and the receptor, a 12 A³ grid box was generated making sure that the Cys25 would be in that grid box. Then the non-covalent docking as described above was carried out followed by the covalent linking and finally minimizing the complex in OPLS force field. Selected examples of docking poses of identified covalent inhibitors 401 and 402 of cysteine protease Cathepsin L are shown in FIGS. 4A-B [Vinyl sulfone 401 and vinyl sulfone 402). The docked poses and the energetics were analyzed and after the analysis, it was found that out of 1648 ligands, only 33 of them exhibit good docking poses with lower complex energies and having docking scores ≥6.0. The docking score of ≥6.0 translates to 1.0 μM of biological activity. These 33 compounds had been recommended for synthesis followed by biological testing.

### Example 13

Experimental validation of identified inhibitors of cysteine protease cathepsin L. Identified covalent inhibitors (including 53) had been synthesized and tested in cathepsin L assays using commercially available fluorescent substrates for cathepsins (Calbiochem). Compound 53 has a Ki of 134±2 μM.

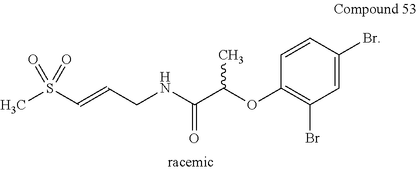 [SMILES: CC(Oc1ccc(Br)cc1Br)C(=O)NC/C=C/S(C)(=O)=O]

Identified covalent inhibitor of cathepsin L (compound 53) from virtual docking screen does not inhibit related Cathepsin B cysteine protease, which suggests it is a selective inhibitor. Detailed kinetic analysis returned K_{inact}/Ki values for compound 53 as 4.1 M⁻¹ s⁻¹ for cathepsin L which is within a range of values reported by us for cysteine protease papain (J. Med. Chem. 2014, 57, 4969-4974).

### Example 14

The binding of the covalent inhibitors designed using present method to the receptor (i.e., papain, a cysteine protease) may be verified by using Mass Spectrometry methods (J. Med. Chem., 2014, 57 (11), pp 4969-4974). k_{inact}/Ki values for known papain inhibitor compounds and papain inhibitors arising out of the present methods are shown in the table below.

| Known inhibitors of papain, k_{inact}/Kᵢ, M⁻¹ s⁻¹ | | Inhibitors of papain in this work, k_{inact}/Kᵢ, M⁻¹ s⁻¹ | |
|---|---|---|---|
| 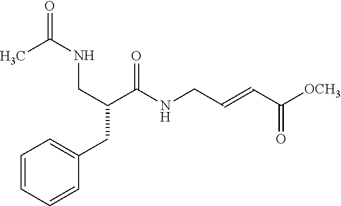 [SMILES: COC(=O)/C=C/CNC(=O)[C@@H](CNC(C)=O)Cc1ccccc1] | 6.575 | 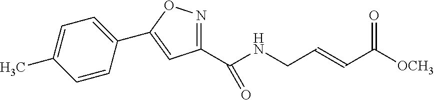 [SMILES: COC(=O)/C=C/CNC(=O)c1cc(-c2ccc(C)cc2)on1] | 1.228 |
| 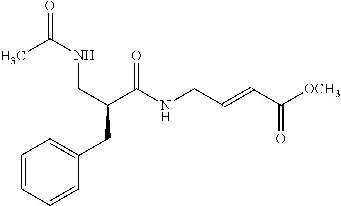 [SMILES: COC(=O)/C=C/CNC(=O)[C@H](CNC(C)=O)Cc1ccccc1] | 0.386 | 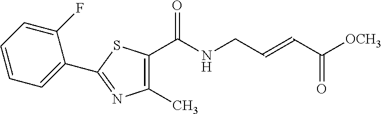 [SMILES: COC(=O)/C=C/CNC(=O)c1sc(-c2ccccc2F)nc1C] | 0.409 |
| 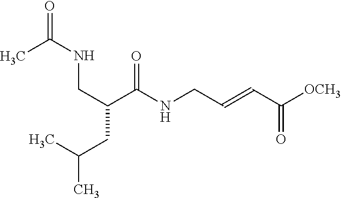 [SMILES: COC(=O)/C=C/CNC(=O)[C@@H](CNC(C)=O)CC(C)C] | 1.102 | 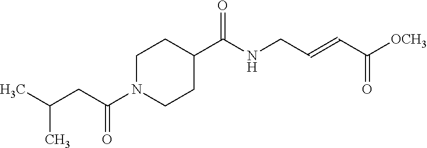 [SMILES: COC(=O)/C=C/CNC(=O)C1CCN(C(=O)CC(C)C)CC1] | 0.461 |
| | | 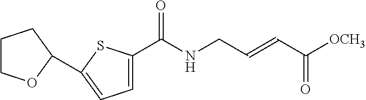 [SMILES: COC(=O)/C=C/CNC(=O)c1ccc(C2CCCO2)s1] | 0.037 |

The present solution may be embodied in other specific forms without departing from its spirit or essential characteristics. The described embodiments are to be considered in all respects only as illustrative and not restrictive. The scope of the present solution is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present solution should be or are in any single embodiment of the invention. Rather, language referring to the features and advantages is understood to mean that a specific feature, advantage, or characteristic described in connection with an embodiment is included in at least one embodiment of the present solution. Thus, discussions of the features and advantages, and similar language, throughout the specification may, but do not necessarily, refer to the same embodiment.

Furthermore, the described features, advantages and characteristics of the present solution may be combined in any suitable manner in one or more embodiments. One skilled in the relevant art will recognize, in light of the description herein, that the present solution can be practiced without one or more of the specific features or advantages of a particular embodiment. In other instances, additional features and advantages may be recognized in certain embodiments that may not be present in all embodiments of the present solution.

Reference throughout this specification to “one embodiment”, “an embodiment”, or similar language means that a particular feature, structure, or characteristic described in connection with the indicated embodiment is included in at least one embodiment of the present solution. Thus, the phrases “in one embodiment”, “in an embodiment”, and similar language throughout this specification may, but do not necessarily, all refer to the same embodiment.

As used in this document, the singular form “a,” “an,” and “the” include plural references unless the context clearly dictates otherwise. The term “(s)” following a noun contemplates the singular or plural form, or both. The term “and/or” means any one of the items, any combination of the items, or all of the items with which this term is associated.

As used in this document, the term “comprising” means “including, but not limited to.” The terms “comprising,” “having,” and “including” are synonymous, unless the context dictates otherwise. The phrases “in one embodiment,” “in various embodiments,” “in some embodiments,” and the like are used repeatedly. Such phrases do not necessarily refer to the same embodiment, but they may unless the context dictates otherwise.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should, therefore, be determined not with reference to the above description, but should instead be determined with reference to the appended claims, along with the full scope of equivalents to which such claims are entitled. The disclosures of all articles and references, including patent publications, are incorporated herein by reference.

Unless defined otherwise, all technical and scientific terms used herein have the same meanings as commonly understood by one of ordinary skill in the art. The features and functions disclosed above, as well as alternatives, may be combined into many other different systems or applications. Various presently unforeseen or unanticipated alternatives, modifications, variations or improvements may be made by those skilled in the art, each of which is also intended to be encompassed by the disclosed embodiments.

What is claimed is:

1. A method for screening libraries of covalent inhibitors, comprising:
(a) providing a potential receptor containing at least one nucleophilic group;
(b) providing a potential covalent inhibitor containing at least one electrophilic group capable of forming a covalent bond with the receptor;
(c) docking the potential covalent inhibitor into the receptor;
(d) ranking the docked potential covalent inhibitor based on one or more screening criteria and their combinations thereof; and
(e) selecting the highly ranked potential covalent inhibitors.

2. The method of claim 1, wherein the one or more screening criteria are selected from the group consisting of Van der Waals radius, angle of attack, solvation effects, cone angle, bond length, bond strength, and electronic character.

3. The method of claim 1, wherein providing the potential receptor further comprising providing three-dimensional structure information of the potential receptor.

4. The method of claim 1, wherein docking the potential covalent inhibitor into the receptor further comprising docking the potential covalent inhibitor into a ligand binding region or a ligand binding pocket of the receptor.

5. The method of claim 1, wherein docking the potential covalent inhibitor into the receptor is performed using a docking algorithm.

6. The method of claim 5, wherein the docking algorithm is GLIDE or CovDock.

7. The method of claim 1, further comprising experimentally testing the selected covalent inhibitors.

8. The method of claim 7, wherein experimentally testing the selected covalent inhibitors further comprising:
measuring binding kinetics of the potential covalent inhibitor, wherein a two-step mechanism specifies a binding kinetics according to the following equation: 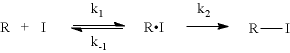
where R is the receptor, I is the potential covalent inhibitor, k₁ is an association constant; k₋₁ is a dissociation constant, and k₂ is an association constant of the receptor that is covalently modified by the potential covalent inhibitor,
wherein the potential covalent inhibitor initially binds reversibly to the receptor, then an electrophilic center of the potential covalent inhibitor binds irreversibly to a nucleophile center of an amino acid within the receptor.

9. The method of claim 1, wherein the potential covalent inhibitor is:
a carboxylic acid (R₁—COOH) coupled to an electrophilic fragment terminated with an aminomethyl group; or
an aminomethyl group (R₁—CH₂—NH₂) coupled to an electrophilic fragment terminated with a carboxylic acid;
wherein R₁ is a drug-like fragment, or a fragment comprising 10-17 non-hydrogen atoms.

10. The method of claim 9, wherein R₁ does not influence a reactivity of an electrophile center of the potential covalent inhibitor with a nucleophile of the receptor.

11. A method of generating a covalent inhibitor library, comprising:
(a) identifying a natural ligand capable of covalently binding to a receptor, wherein the natural ligand comprising a directing group and a reactive group containing an electrophilic fragment;
(b) modifying the reactive group by replacing the directing group with a carboxylic acid or an amine; and
(c) coupling the carboxylic acid-modified reactive group to a second directing group comprising an aminomethyl group (R₁—CH₂—NH₂); or
(d) coupling the amine-modified reactive group to a third directing group comprising a carboxylic acid.

12. The method of claim 11, wherein the electrophilic fragment is a Michael acceptor or an alkylating agent.

13. The method of claim 11, wherein the electrophilic fragment is an sp² Michael acceptor or an sp Michael acceptor.

14. The method of claim 11, wherein the sp² electrophilic fragment is selected from the group consisting of:

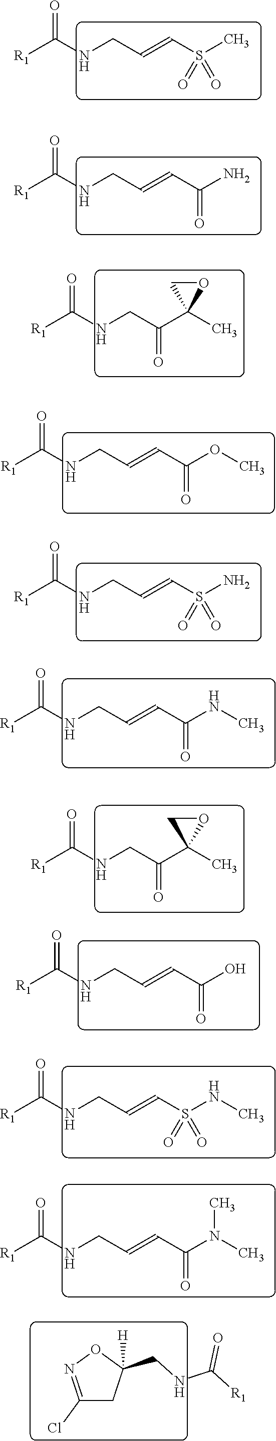

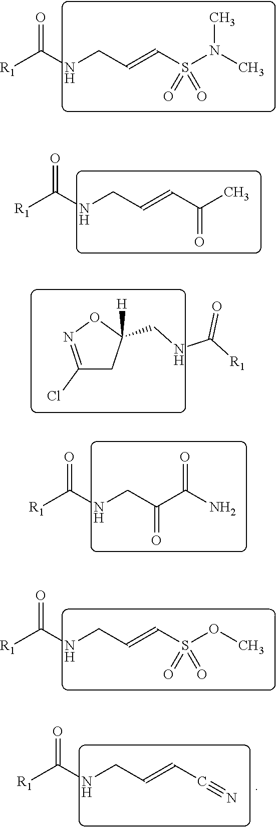

15. The method of claim 11, wherein the sp electrophilic fragment is selected from the group consisting of:

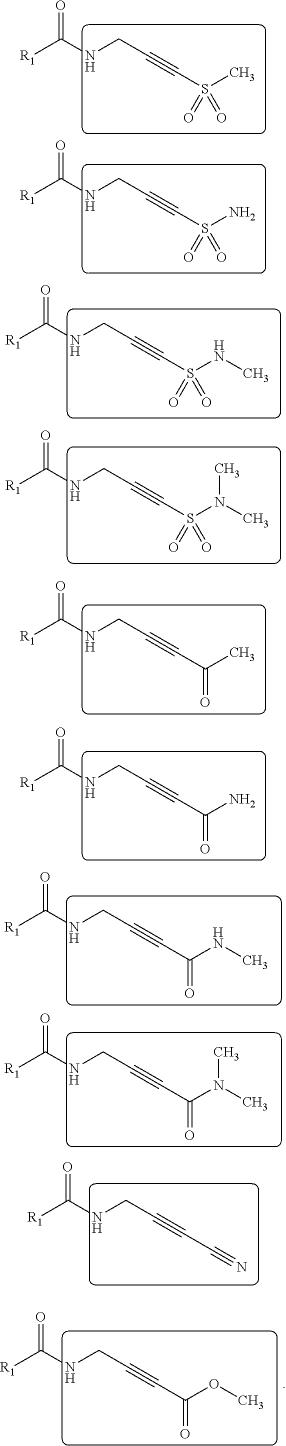

16. The method of claim 11, wherein the electrophilic fragment further comprises an electron withdrawing group.

17. The method of claim 16, wherein the electron withdrawing group is selected from the group consisting of nitros, amides, esters, acid, nitriles, ketones, sulfones, sulfoxides, sulfonamides, nitriles, halides, lactams, lactones, oxygen heterocycles, nitrogen heterocycles, substituted or unsubstituted aromatic fragments, and epoxides.

18. The method of claim 17, wherein the potential covalent inhibitor is an agonist, an inverse agonist, an antagonist or a neutral antagonist.

19. The method of claim 17, wherein an electrophile center of the potential covalent inhibitor reacts with a nucleophile center of an amino acid in the receptor.

20. The method of claim 19, wherein the nucleophile center of the amino acid in the receptor is sulfur or nitrogen.

21. The method of claim 19, wherein the amino acid is cysteine, methionine, proline, tryptophan, asparagine, glutamine, tryptophan, or histidine.

22. The method of claim 11, wherein the aminomethyl-coupled electrophilic fragment is selected from the group consisting of: 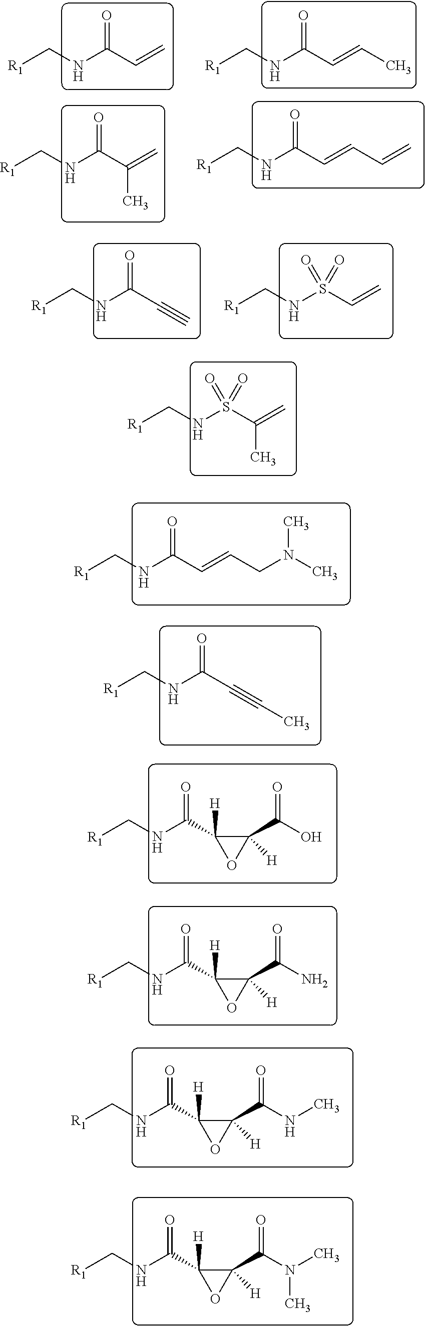 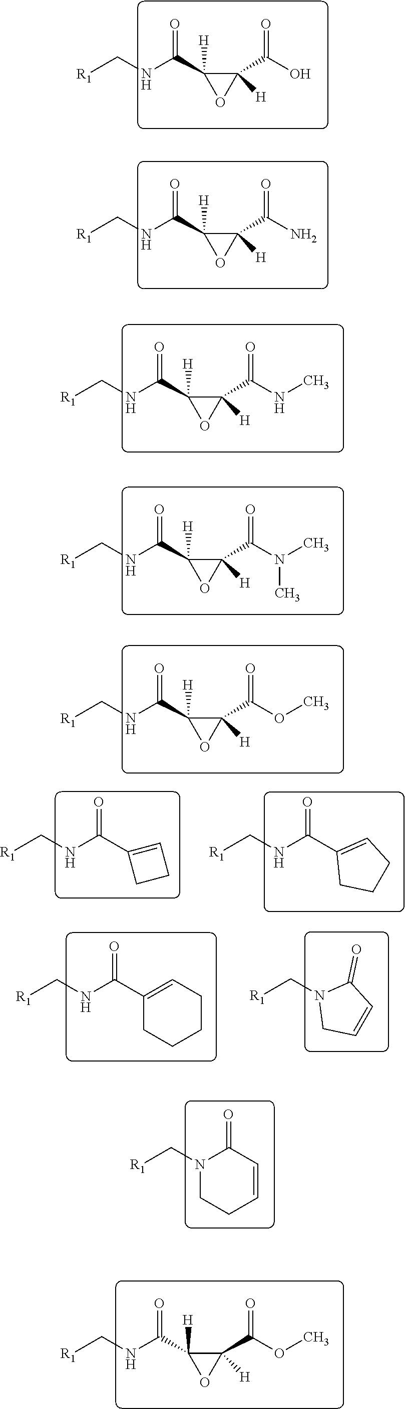
wherein R₁ is a drug-like fragment, or a fragment comprising 10-17 non-hydrogen atoms.

23. A covalent inhibitor generated from the method of claim 22.
